(19) United States
(12) Patent Application Publication
(10) Pub. No.: US 2005/0244893 A1
(43) Pub. Date: Nov. 3, 2005

(54) Novel method for determination of plasminogen activator inhibitor

(75) Inventors: David L. Crandall, Doyleston, PA (US); Duane E. Day, Novi, MI (US)

Correspondence Address: WILMER CUTLER PICKERING HALE AND DORR LLP, 60 STATE STREET, BOSTON, MA, 02109, US

(73) Assignees: Wyeth, Madison, NJ (US); Molecular Innovations, Inc., Southfield, MI (US)

(21) Appl. No.: 11/106,904

(22) Filed: Apr. 15, 2005

Related U.S. Application Data
(60) Provisional application No. 60/563,130, filed on Apr. 16, 2004.

(51) Int. Cl. G01N 33/53

(52) U.S. Cl. 435/7.1

(57) ABSTRACT

The invention provides a novel assay system for measuring the amount of active PAI-1 in a sample with sensitivity, and correlation to an active PAI-1 amount. The assay determines the amount of active PAI-1 in a sample by utilizing a novel standard curve. It is emphasized that this abstract is provided to comply with the rules requiring an abstract that will allow a searcher or other reader to quickly ascertain the subject matter of the technical disclosure. It is submitted with the understanding that it will not be used to interpret or limit the scope or meaning of the claims. 37 CFR 1.72(b).

### CROSS-REFERENCE TO RELATED APPLICATIONS

This application claims priority to provisional U.S. application Ser. No. 60/563,130 filed on Apr. 16, 2004, which is herein incorporated by reference in its entirety.

### FIELD OF THE INVENTION

This invention is in the field of biochemical assay systems. More specifically, this invention relates to the detection and quantitation of Plasminogen Activator Inhibitor One (PAI-1).

### BACKGROUND OF THE INVENTION

Plasminogen activator inhibitor one (PAI-1) is a major regulatory component of the plasminogen-plasmin system. Further, it is the principal physiologic inhibitor of both tissue type plasminogen activator (tPA) and urokinase type plasminogen activator (uPA). Elevated plasma levels of PAI-1 have been associated with thrombotic events as indicated by animal experiments (Krishnamurti, Blood 69:798 (1987); Carmeliet, J. Clin. Invest. 92:2756 (1993); Farrehi, Circulation 97:1002 (1998)) and clinical studies (Juhan-Vague, Thrombosis and Haemostasis, 57:67 (1987)). Antibody neutralization of PAI-1 activity resulted in promotion of endogenous thrombolysis and reperfusion (Biemond, Circulation 91:1175 (1995); Levi, Circulation 85:305 (1992)). Elevated levels of PAI-1 have also been implicated in diseases of women such as polycystic ovary syndrome (Nordt, J. Clin. Endocrinol. Metabol. 85(4):1563 (2000)) and bone loss induced by estrogen deficiency (Daci, J. Bone Min. Res. 15:1510 (2000)). High PAI-1 mass concentrations are reported to be associated with increased risk of myocardial infarction in both men and women (Thogersen et al., Circulation 98:2241-2247 (1998)). Furthermore, a high level of PAI-1 is indicative of cardiovascular disorders such as atherosclerosis, deep vein thrombosis, and Type 2 diabetes mellitus (Juhan-Vague et al., Circulation, 94:2057-2063 (1996)). Plasma PAI-1 is also elevated in postmenopausal women, and has been proposed to contribute to the increased incidence of cardiovascular disease in this population (Koh, N. Engl. J. Med. 336:683 (1997)). Moreover, there exists a primary role of PAI-1 in thrombus stabilization, smooth muscle cell migration, and cardiac fibrosis. PAI-1 stabilizes both arterial and venous thrombi, contributing respectively to coronary arterial occlusion in post-myocardial infarction (Hamsten, Lancet 2:3 (1987)) and venous thrombosis following post-operative recovery from orthopedic surgery (Siemens, J. Clin. Anesthesia 11:622 (1999)). Deficiencies in plasminogen activator inhibitor-1 (PAI-1) result in blood related disorders such as delayed, or prolonged, bleeding see, for example, Reilly et al. (Blood Coag. Fibrinolysis 5:73-81 (1994)). In addition, studies have been published on the prognostic value of PAI-1 in cancer, e.g., pancreatic cancer (Albo et al., J. Gastrointestinal Surg. 3:411-417 (1999)), ovarian cancer (Borgfeldt et al., Int. J. Cancer 92:497-502 (2001)) and in breast cancer (Foekens et al., Cancer Res. 60:636-643 (2000)).

PAI-1 exhibits the general serpin property of conformational plasticity. Multiple conformational states of PAI-1, including active, latent, and cleaved forms were described in 1997 (Lawrence, Nat. Struct. Biol. 4:339 (1997)). Because of structural instability, PAI-1 in human plasma is found in highest concentrations in its inactive or latent state. However, while active PAI-1 is structurally unstable with a plasma half-life of approximately one hour, it is the active conformation that is inhibitory toward tPA and uPA, and therefore the conformation believed to be associated with the development of various PAI-1 related disorders, inter alia, cardiovascular disease (Vaughan, J. Invest. Med. 46:370 (1998)).

Elevated PAI-1 during acute peripheral arterial thrombolysis is associated with an increased risk of lysis failure due to reduced levels of circulating active tPA or urokinase (Nicholls et al., Blood Coagulation Fibrinolysis 14(8):729-733 (2003)). The hallmark of failed thrombolytic therapy for acute peripheral arterial thrombosis is the presence of measurable PAI-1 activity during the infusion of the thrombolytic agent. The thrombolytic agent should neutralize all the PAI-1 present with the excess available to lyse the thrombus. When an excess of thrombolytic agent is not given, only a portion of the PAI-1 is neutralized, the majority of the thrombolytic agent is bound to PAI-1 and thrombolysis fails. In one study, tPA infusion in the failed lysis group, only 3% of the circulating tPA was in the active form versus 45% active tPA in the successful lysis group. In the failed lysis group, endogenously secreted PAI-1 was inhibiting the tPA approximately as rapidly as it was being infused. It was suggested that one potential solution for the problem of high PAI-1 neutralizing infused tPA or urokinase would be the rapid and accurate measurement of PAI-1 activity during thrombolytic therapy. Since therapy often lasts for up to 24 hours, time would be available for monitoring if a suitable assay were available. The dose of thrombolytic agent could then be adjusted to overcome the PAI-1 activity present. Monitoring of PAI-1 activity and thrombolytic dose during therapy could also reduce bleeding if lower doses of thrombolytic agent could be used in patients with low baseline PAI-1 activity. While minor hemorrhagic complications are generally well tolerated, major hemorrhagic complications including retroperitoneal and intracranial hemorrhage can be fatal. Monitoring of PAI-1 prior to and during thrombolysis, and adjustment of the dose of thrombolytic agent may both increase the probability of successful peripheral arterial thrombolysis and reduce incidence of hemorrhage. To perform this type of monitoring, a rapid, accurate PAI-1 assay or rapid measure of plasminogen activator activity is needed. Most current PAI-1 activity and plasminogen activator activity assays are designed to be run in batches and are not optimized for single rapid measurements.

In summary, currently available clinical diagnostic assay systems for plasma PAI-1 suffer from one or more of the following disadvantages:

1. The assays do not specifically address the metastable property of PAI-1, preventing accurate determination of the active conformation PAI-1. For example, Coaliza® PAI-1 (Chromogenix; Milano, Italy), TintElize® and Imulyse® (Trinity Biotech; Wicklow, Ireland), and Zymutest PAI-1 Antigen™ (Hyphen BioMed, Andresy, France) measure total PAI-1 antigen.

2. Available assays can require denaturing and renaturing of PAI-1; and/or have a low degree of reproducibility. For example, the Biopool diagnostic assay (Chromolize®) uses a standard consisting of PAI-1 generated from a human cancer cell line that is reactivated and Zymutest PAI-1 Activity™ uses a recombinant wild type PAI-1 produced in E. coli. The PAI-1 produced by this method is predominantly in the latent form and requires harsh renaturation protocols to recover partial biological activity. Generally, strong denaturants such as sodium dodecyl sulfate, guanidine HCl, and urea are used to reactivate latent PAI-1 into an active conformation (Lambers et al., J. Biol. Chem. 262(36):17492-17496 (1987)). Such denaturants typically result in a maximum recovered activity of 40-60% (Stromqvist, et al., Protein Expr. Purif. 5(4):309-316 (1994)). In addition, the renaturation process would be expected to result in batch to batch variability. Therefore, each lot of assay kits must be standardized against an international reference standard which itself has been produced by the same methodology. As a result, the clinical studies using these assays have shown considerable assay variability. Additionally, because production of the international standard itself lacks reproducibility, once these international standard stocks have been depleted, existing assay kits or newer kits in development will lack a reliable control.

3. Multiple measurement steps are required. Coatest® PAI-1 (Chromogenix, Milano Italy), Spectrolize®/pL PAI-1 and Spectrolize®/Fibrin (Trinity Biotech, Wicklow, Ireland), each utilize a two-stage indirect assay whereby a fixed amount of tPA is added to the sample to be analyzed. Active PAI-1 present in the sample forms a complex with the tPA. The residual tPA is then used to activate plasminogen to plasmin in the presence of a tPA stimulator. The amount of plasmin formed is proportional to the residual tPA and inversely proportional to the PAI-1 present in the original sample. Plasmin activity is then measured and the PAI-1 activity calculated. This assay measures PAI-1 activity indirectly and requires measurement of the tPA activity, and also requires a number of components and steps. These assays also require two separate measurements of the tPA before and after incubation with the sample to be analyzed.

4. At least some assays limited sensitivity-Chromolize® PAI-1 and Zymutest PAI-1 Activity™ (Hyphen BioMed) use a monoclonal horseradish peroxidase (HRP) conjugate, which potentially limits sensitivity.

5. The commercially available kits provide a PAI-1 activity standard of 50 IU/ml or lower. For example, Biopool's Chromolize® kit provides a maximum PAI-1 activity standard of 50 IU/ml requiring a repeat of the assay by dilutions into PAI-1-depleted plasma if the plasma PAI-1 exceeds the standard. This results in the consumption of both reagents and time.

A significant advance would be an assay system that can accurately and rapidly measure low or high levels of active PAI-1 in vivo as well as in vitro. For example, accurate measurement of PAI-1 levels would be useful as a prognostic or diagnostic marker with respect to occurrence of PAI-1 related disorders. More specifically, methods that could accurately measure active PAI-1 would be of utility in diagnosing conditions originating from various PAI-1 related disorders such as disorders associated with abnormalities in hemostatsis and fibrinolysis (e.g., deep vein thrombosis, coronary heart disease, pulmonary embolism, and polycystic ovary syndrome). Additionally, it is highly desirable to provide a PAI-1 standard that is fully active and whose biological activity can be accurately and simply quantitated without an outside calibrator, and one that is produced with a very high degree of reproducibility is highly desirable.

### SUMMARY OF THE INVENTION

The invention is related to the development of a method for accurate determination of active PAI-1, e.g., using a stable mutant of PAI-1 that is fully active as a standard.

Accordingly, the invention relates to a method that includes providing a STABLY ACTIVE PAI-1, and establishing a standard curve using the STABLY ACTIVE PAI-1. In some embodiments, the method includes comparing the amount of PAI-1 in a sample to the standard curve. The half-life of the STABLY ACTIVE PAI-1 is, in some cases greater that the half-life of a wild type PAI-1, e.g., at least two-fold greater than the half-life of a wild type PAI-1. The STABLY ACTIVE PAI-1 can be derived from a wild type PAI-1 and contain a mutation, e.g., at least one of K154T, Q319L, M354I, N150H, and combinations thereof. In some embodiments, the mutation is K154T, Q319L, M354I, or N15OH; or the STABLY ACTIVE PAI-1 includes all of these mutations. In some embodiments of the invention, the is a biological sample, e.g., the sample is from a human. The sample can be, e.g., a body fluid such as blood, plasma, serum, or urine.

In another aspect, the invention is a method for determining the amount of active plasminogen activator inhibitor-1 (PAI-1) in a sample, in which the method includes providing a sample; contacting the sample with a PAI-1 binding molecule, thereby forming an active PAI-1 complex comprising a PAI-1 binding molecule and an active PAI-1; separating the active PAI-1 complex from uncomplexed components; and determining the amount of active PAI-1 in the sample by detecting the amount of the active PAI-1 complex and correlating the amount of the active PAI-1 complex to the amount of active PAI-1 using a standard curve. In yet another aspect, the invention is a method for determining the amount of active PAI-1 in a sample in which the method includes providing a sample; contacting the sample with a PAI-1 binding molecule, and an anti-plasminogen activator-1 inhibitor antibody (anti-PAI-1) to form a PAI-1 complex comprising the antibody, active PAI-1 from the sample, and the PAI-1 binding molecule; separating the PAI-1 complex from an uncomplexed components; and determining the amount of active PAI-1 in the sample by detecting the amount of PAI-1 complex and correlating the amount of complex with the amount of active PAI-1 using a standard curve. In methods of the invention in which a PAI-1 binding molecule is included, the PAI-1 binding molecule can be immobilized on an insoluble support, e.g., immobilized directly onto an insoluble support. In some cases, the PAI-1 binding molecule is chemically modified to be immobilized onto the insoluble support. In some embodiments in which an antibody is used in a method, the PAI-1 complex is separated from unbound antibody prior to detecting the amount of PAI-1 complex. In some cases, a PAI-1 binding molecule/PAI-1 complex formed and is separated from unbound components prior to forming the PAI complex with the anti-PAI-1. The PAI-1 binding molecule is, in some cases, immobilized on an insoluble support via one or more linker molecules. The linker molecule can include an antibody that can bind the PAI-1 binding molecule. In some aspects an antibody is immobilized onto the insoluble support via a secondary linker molecule, e.g., a linker that includes a reporter group such as a radioisotope, a fluorescent group, a luminescent group, an enzyme, biotin, a dye particle, and combinations thereof. In certain embodiments, the secondary linker molecule is selected from the group consisting of avidin and biotin. The detecting of aspects of the invention can be detecting using an enzyme-linked immunosorbent assay (ELISA), a Western blot, an immunohistochemical assay, an immunofluorescence assay, or an imaging assay.

In certain embodiments of the invention in which a PAI-1 binding molecule is used, the PAI-1 binding molecule is a serine proteinase, e.g., a plasminogen activator such as a urokinase plasminogen activator or a tissue type plasminogen activator. The PAI-1 binding molecule can, in some cases, be a serine protease, tPA, uPA, vitronectin, glycosaminoglycan, fibronectin, cathepsin G, prostate specific antigen, and combinations thereof. An antibody used in an invention described herein can be a monoclonal antibody or a polyclonal antibody.

Embodiments of the invention include the methods described herein, in which the standard curve is established using a STABLY ACTIVE plasminogen activator inhibitor-1 (STABLY ACTIVE PAI-1), e.g., a STABLY ACTIVE PAI-1 in which the half-life of the STABLY ACTIVE PAI-1 is greater that the half-life of a wild type PAI-1, for example, at least two-fold greater than the half life of a wild type PAI-1. The STABLY ACTIVE PAI-1 can be derived from a wild type PAI-1 and include a mutation, e.g., the PAI-1 can be derived from a wild type PAI-1 and include at least one mutation that is K154T, Q319L, M354I, N150H, or a combination thereof, or the STABLY ACTIVE PAI-1 contains a mutation that is K154T, Q319L, M354I, or N150H.

The invention also includes a method for determining the amount of active plasminogen activator inhibitor-1 (PAI-1) in a sample, in which the method includes providing a sample; contacting the sample with a PAI-1 binding molecule, thereby forming an active PAI-1 complex comprising a PAI-1 binding molecule and an active PAI-1; and determining the amount of active PAI-1 in the sample by detecting the amount of the active PAI-1 complex and correlating the amount of the active PAI-1 complex to the amount of active PAI-1 using a standard curve, and wherein the standard curve is established using a STABLY ACTIVE PAI-1.

Another aspect of the invention is a method for diagnosing a PAI-1 related disorder in a subject. The method includes obtaining at least one biological sample from a subject; contacting the biological sample with a PAI-1 binding molecule to form an active PAI-1 complex comprising PAI-1 binding molecule and active PAI-1; separating the active PAI-1 complex from a uncomplexed components; and determining the amount of active plasminogen activator inhibitor-1 in the biological sample by detecting the amount of active PAI-1 complex in the sample and correlating the amount of complex to the amount of active PAI-1 using a standard curve. The can be a mammal, e.g., a human. The biological sample can be a body fluid, e.g., blood, plasma, serum, or urine. In some embodiments, the PAI-1 binding molecule is a serine proteinase, e.g., a plasminogen activator such as urokinase plasminogen activator or tissue plasminogen activator. The PAI-1 binding molecule can be, in some cases, tPA, uPA, vitronectin, glycosaminoglycan, fibronectin, cathepsin G, prostate specific antigen, and combinations thereof. In certain embodiments of the method for diagnosing, the standard curve is established using a STABLY ACTIVE PAI-1.

In another aspect, the invention relates to a diagnostic kit that includes components for carrying out a method for determining the amount of PAI-1 in a sample, e.g., a biological sample such as blood, serum, or plasma. In certain embodiments, the diagnostic kit includes a STABLY ACTIVE PAI-1. In another embodiment, the diagnostic kit includes instructions for preparing a standard curve, e.g., using a STABLY ACTIVE PAI-1.

The invention also includes a diagnostic kit that includes a PAI-1 binding molecule; at least one anti PAI-1 binding molecule antibody; at least one detection reagent; and a STABLY ACTIVE plasminogen activator-1 inhibitor. The kit can also include at least one buffer and instructions for use of the kit. In some embodiments, the PAI-1 binding molecule is a serine protease, e.g., a plasminogen activator such as a urokinase plasminogen activator or a tissue plasminogen activator. The kit can contain an insoluble support, e.g., the PAI-1 binding molecule in supplied on the insoluble support.

In another aspect, the invention includes a method of identifying an agent that can modulate active PAI-1. The method includes providing a test agent; determining whether the test agent can modulate the activity of PAI-1 using a method described herein in which a sample is tested and compared to a standard curve; and selecting a test agent that modulates the activity of PAI-1. In some cases, a STABLY ACTIVE PAI-1 is used to establish the standard curve.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In addition, the materials, methods, and examples are illustrative only and not intended to be limiting.

Other features and advantages of the invention will be apparent from the detailed description, drawings, and from the claims.

### BRIEF DESCRIPTION OF THE DRAWINGS

FIG. 1 is a graph depicting a typical standard curve prepared by the addition of the STABLY ACTIVE PAI-1 (CPAI) standard to human plasma that had been immuno-depleted of natural PAI-1 antigen and activity depleted by thermal inactivation.

FIG. 2 is a line graph depicting the results of a thermal stability study in which the activity of three PAI-1 plasma activity standards were compared as a function of time at 37° C. The three samples were STABLY ACTIVE PAI-1 activity standard at 30 U/ml (not standardized against the NIBSC (National Institute for Biological Standards and Control; Potters Bar, UK) activity standard 92/654) in plasma (squares), Biopool Chromolize® activity standard (30 U/ml) in plasma (triangles), and NIBSC 92/654 plasma activity standard (27.5 U/ml) (circles). The activities of each sample were calculated and subsequently plotted by using the standard curve shown in FIG. 1.

FIG. 3a is a graph depicting a standard curve generated using STABLY ACTIVE PAI-1 and standardized against the NIBSC standard.

FIG. 3b is a graph depicting a standard curve produced using the Biopool predicate kit that was prepared according to the kit instructions.

FIG. 4a is a graph depicting the results of experiments in which antibody 6713E5.29 was used to detect STABLY ACTIVE PAI-1 to generate a standard curve.

FIG. 4b is a graph depicting the results of experiments in which antibody 6712A7.8 was used to detect STABLY ACTIVE PAI-1 to generate a standard curve.

FIG. 4c is a graph depicting the results of experiments in which the antibody provided in the HRP-conjugated Biopool kit was used to detect STABLY ACTIVE PAI-1 to generate a standard curve.

FIG. 5 is a bar graph depicting the results of experiments in which the Biopool assay system and the assay system using the STABLY ACTIVE PAI-1 was used to assay different human plasma samples.

FIG. 6 is a photographic reproduction of a gel depicting the purity and activity of STABLY ACTIVE PAI-1. Lane 1 was loaded with PAI-1 and illustrates the purity. Lane 2 was loaded with STABLY ACTIVE PAI-1 sample complexed with an excess of human uPA.

### DETAILED DESCRIPTION OF THE INVENTION

Applicants have succeeded in developing a novel assay that allows a rapid, accurate, and reproducible measurement of active plasminogen activator inhibitor type 1 (PAI-1) in vivo as well as in vitro by utilizing a STABLY ACTIVE mutant form of PAI-1 as a standard.

The novel assay method of the present invention has one or more of the following advantages over existing PAI-1 assay systems: (1) The kit of the present invention described herein utilizes a one-step direct assay that measures bound PAI-1 and requires only one assay to determine PAI-1 activity. (2) The assay Captures only active PAI-1. (3) The assay employs a monoclonal detection followed by detection with an HRP polyclonal secondary conjugate that increases the assay sensitivity. (4) The method is highly reproducible since it utilizes a superior PAI-1 activity standard (stable mutant) to construct the calibration curve. (5) Batch to batch standards are essentially 100% active and can be independently standardized without the use of an international standard. (6) Since the STABLY ACTIVE PAI-1 is 100% active, the PAI-1 activity does not have to be standardized against another calibrator. (7) For the PAI-1 preparation of high purity (as in the present invention), the protein concentration can be accurately determined. The active PAI-1 concentration is then equivalent to the protein concentration. Accordingly, the results of the assay system of the present invention are in an excellent dynamic range and linearity. (8) The novel assay system and kit of the present invention provides linearity beyond even 100 U/ml.

In one embodiment, plasminogen activator is immobilized on an insoluble support. Samples containing active PAI-1 are added. Active PAI-1 present in the sample reacts with plasminogen activator coated on a insoluble support. Latent or complexed PAI-1 does not bind to the plasminogen activator and will not be detected. After appropriate washing steps, anti-PAI-i primary antibody is added to the plate and binds to PAI-1. Excess antibody is washed away and bound antibody, which is proportional to the original active PAI-1 present in the plasma sample, is then reacted with the secondary antibody. A labeled second antibody, immuno-specific for the first antibody, may be used as the indicating method. For example, the secondary antibody can be conjugated to horseradish peroxidase. A substrate such as TMB substrate (3,3′,5,5′-tetramethylbenzidine) is then used for color development that is detected at 450 nm. A standard calibration curve is prepared using a novel, stable mutant of active PAI-1 (STABLY ACTIVE PAI-1) in PAI-1 activity depleted plasma. Plasminogen activator can be tPA or uPA. uPA can be tc uPA or sc uPA. tPA can be tc tPA or sc tPA.

Generation of a standard curve using STABLY ACTIVE PAI-1 is described herein. Such standard curves are useful for PAI-1 assays and have, as presented herein, advantages over methods described in the art. For example, such a curve can be generated using an E. coli produced recombinant mutant PAI-1 (a STABLY ACTIVE PAI-1) that is isolated as described in WO200383104 and U.S. patent application Ser. No. 10/370,828. Standard curves are prepared using the STABLY ACTIVE form of PAI-1. Serial dilutions of a recombinant PAI-1 that is STABLY ACTIVE (STABLY ACTIVE PAI-1) in a PAI-1 depleted human plasma sample are prepared. Dilutions typically cover a range between 0 and 150 ng/ml (e.g., 0, 10, 20, 50, 100, 150 ng/ml). The range of 0 to 150 ng/ml is generally adequate to detect plasma PAI-1 in normal individuals (subjects), and/or in patients (subjects) with altered PAI-1 levels. However, the assay of the present invention is easily adaptable to any PAI-1 level (concentration) by preparing additional serial dilutions of the plasma. For example, in a subject for whom plasma PAI-1 may be in excess of 150 ng/ml (e.g., 1000 ng/ml) appropriate serial dilutions of plasma with a buffer or aqueous solution, such as, Tris, HEPES, or NaCl, can easily be prepared and used for PAI-1 detection. PAI-1 concentration is plotted on X-axis (linear scale) and fluorescence or absorbance on the Y-axis (either log or linear scale). The concentration of the PAI-1 in the sample is interpolated using the standard curve.

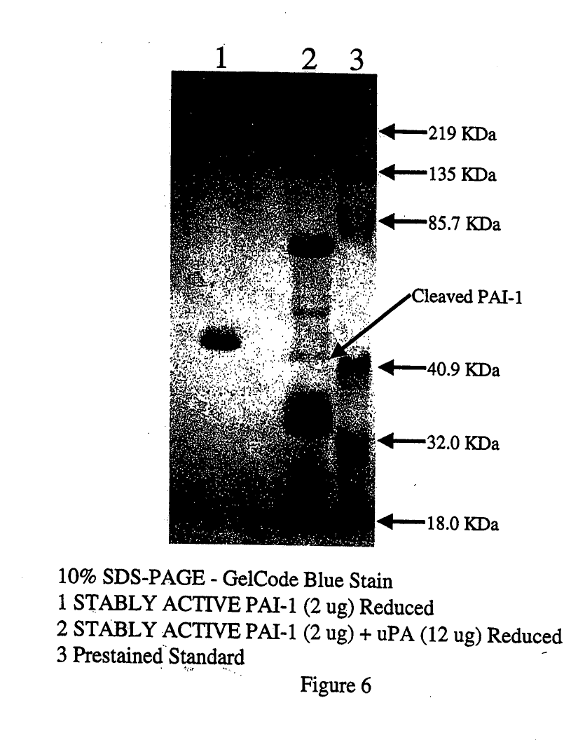

In other embodiments, the present invention uses the STABLY ACTIVE PAI-1 activity standard as an internal reference standard without employing any reference standard such as the NIBSC standard. The STABLY ACTIVE PAI-1 used in aspects of the present invention was examined using 10% polyacrylamide gel electrophoresis (PAGE) and was found to be highly pure (e.g., 99% pure) (FIG. 6, lane 1). To determine the stability of STABLY ACTIVE PAI-1, a preparation of STABLY ACTIVE PAI-1 was mixed with urokinase and analyzed using PAGE. The result demonstrates that the PAI-1 was fully active (FIG. 6, lane 2). In these data, it can be seen that all of the PAI-1 migrates into complex with the active site containing chain of urokinase with less than 5% of the STABLY ACTIVE PAI-1 forming a nonproductive cleaved byproduct that is a normal process of the bifurcation pathway for PAI-1 inhibition. Thus, STABLY ACTIVE PAI-1 scan serve as its own internal reference standard. One can determine the concentration STABLY ACTIVE PAI-1 by methods well known in the art (e.g., spectral measurements or sequence analysis) and prepare a standard curve for use in assays in which it is desirable to determine the amount of active PAI-1 in a sample, e.g., a biological sample or a synthesized sample.

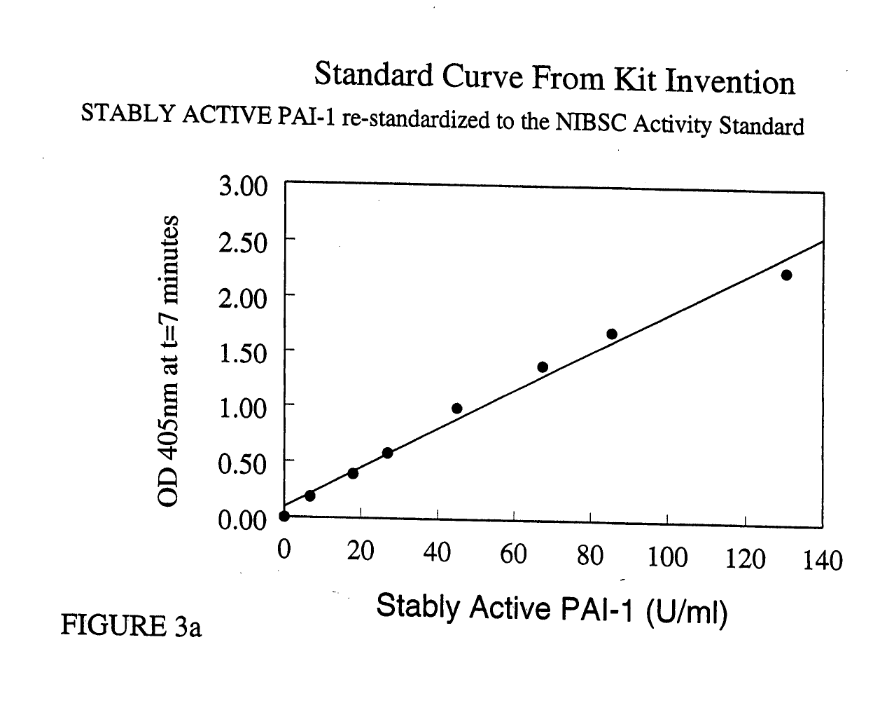

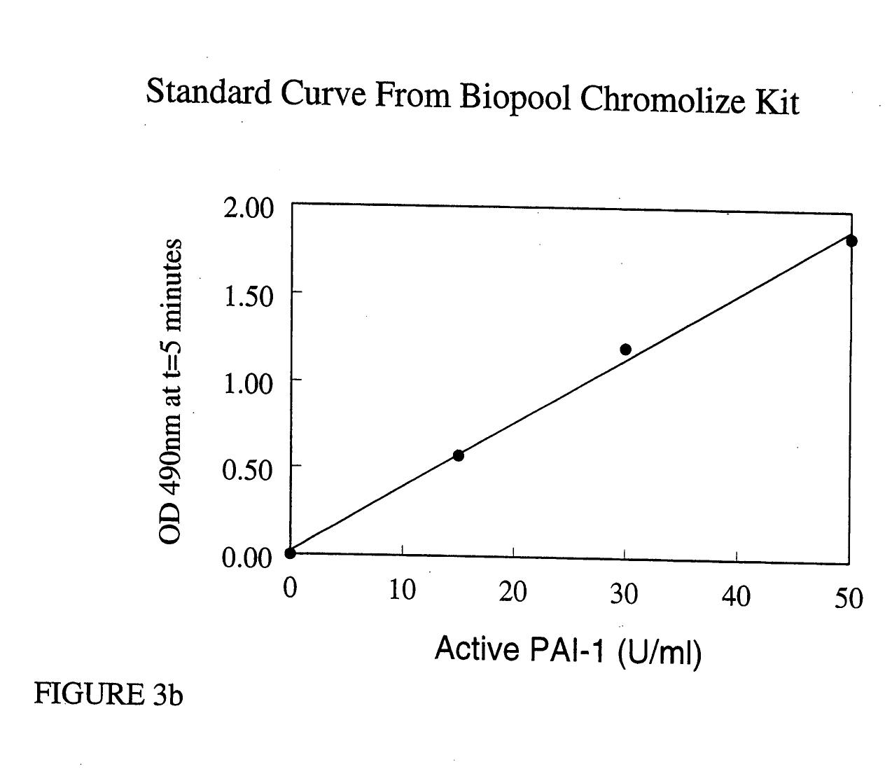

A PAI-1 activity unit is generally defined as the amount of activity that will neutralize 1 IU of tPA activity. Because by international agreement 1 mg of recombinant glycosylated single chain human tPA contains 600,000 IU, the molecular weight of tPA is 64,000 kDa, and the molecular weight of E. coli produced non-glycosylated PAI-1 is about 43,000, then by calculation, 1 U of PAI-1 activity corresponds to 1.15 ng of active PAI-1. The predicate Biopool Chromolize® kit is standardized against the NIBSC PAI-1 activity standard 92/654. This NIBSC activity standard is supplied as a lyophilized sample under vacuum in the Biopool kit. When the sample is reconstituted in 1 ml of purified water, the contents are reported to contain 27.5 U/ml of PAI-1 activity. When the concentration of PAI-1 activity of the NIBSC standard is measured with an assay (e.g., that can be provided in a kit) using the STABLY ACTIVE PAI-1 as an internal control standard, then the calculated values are consistently lower than expected. For example, FIG. 3 a shows a typical standard curve (e.g., as from a kit of the present invention) using the STABLY ACTIVE PAI-1 in plasma. The curve shows the standard curve using the STABLY ACTIVE PAI-1 standard as an internal calibrator. In this instance the NIBSC standard reports a calculated value of 18 U/ml PAI-1 activity. The same data are shown in FIG. 3b, but the PAI-1 concentration was re-calibrated using the 27.5 U/ml NIBSC activity standard as an external reference. Upon correction the NIBSC standard had a calculated value of 31 U/ml. The Biopool kit used with all Biopool components resulted in a calculated value of 26 U/ml for the NIBSC standard (FIG. 3b).

The NIBSC standard itself suffers from precision and reproducibility. The NIBSC standard is prepared from recombinant human PAI-1 produced in a Chinese Hamster Ovary cell line. This PAI-1 is produced in the latent form and is subjected to harsh reagents for reactivation. It is not clear if this activity of the NIBSC standard was determined before or subsequent to lyophilization, which may affect activity. Furthermore, due to the labile nature of NIBSC activity standard, the long-term reproducibility of the standard becomes questionable. Accordingly, the use of the NIBSC standard will, in general, result in an overestimation of the true active concentration of PAI-1 activity in a given biological sample. However, use of the STABLY ACTIVE PAI-1 as the standard can solve the above problems and provides a more quantitative result. Thus, an aspect of the invention is a standard curve generated using STABLY ACTIVE PAI-1. Such a standard curve can be used for determining the amount of PAI-1 in a sample.

In other embodiments, the present invention provides for an assay method for determining the amount of active PAI-1 in a sample, the method comprising a) contacting the sample with a PAI-1 binding molecule to form an active PAI-1 complex comprising PAI-1 binding molecule and active PAI-1; b) separating the active PAI-1 complex from an uncomplexed components, c) determining the amount of active plasminogen activator inhibitor-1 in the sample by detecting the amount of the active PAI-1 complex and correlating the amount of complex to the amount of active PAI-1 from a standard curve (e.g., a standard curve generated using STABLY ACTIVE PAI-1).

Accordingly, it is possible to derive from the values generated using certain assays described herein, the amount of active PAI-1 present in a biological sample. This can be achieved by comparing the results obtained using the biological samples with a standard curve (e.g., a standard curve obtained using STABLY ACTIVE PAI-1). Therefore, a method provided herein can further comprise calculating the amounts of [(PAI-binding molecule)-(PAI-1)] complex from the respective values by comparing the respective values with corresponding values on a standard curve, the standard curve being a plot of known concentrations of [(PAI-binding molecule)-(STABLY ACTIVE PAI-1)] complex against the corresponding value of detectable marker.

The standard curve described herein can be obtained by carrying out with at least one reference sample comprising a specific known concentration of [(PAI-binding molecule)-(STABLY ACTIVE PAI-1)] complex and/or one or more dilutions of the reference sample, thereby obtaining two or more values corresponding to two specific concentrations of [(PAI-binding molecule)-(STABLY ACTIVE PAI-1)] complex and plotting the determined values against their respective known concentrations. The known concentration of [(PAI-binding molecule)-(STABLY ACTIVE PAI-1)] can be provided by the manufacturer of the reference sample, or can be determined independently.

A PAI-1 binding molecule can be attached to an insoluble support directly or indirectly (e.g., via a linker molecule) for use in the assay to determine and/or measure the presence of a PAI-1 in a biological sample. In one embodiment, an antibody that can specifically bind a PAI-1 binding molecule is immobilized on an insoluble support, followed by the addition the PAI-1 binding molecule. This combination of additions results in the PAI-1 binding molecule being bound to the insoluble support indirectly via an antibody, yet still functionally capable of capturing active PAI-1 from an added biological sample. Accordingly, such an antibody will recognize PAI-1 binding molecule, mutants, fragments and/or combinations thereof upon contact. The sample is then contacted with PAI-1 binding molecule bound to the immobilized antibody on an insoluble support and unbound components are removed from components bound to the insoluble support. The insoluble support is contacted with an anti-plasminogen activator-1 inhibitor antibody to form antibody-antigen complexes. The unbound anti-plasminogen activator-1 antibody is removed and antibody-antigen complexes bound to the insoluble support are detected by a detection reagent. The amount of antibody-antigen complex in the biological sample is determined and the measured amounts are correlated with a standard curve. The results are indicative of the amount of active plasminogen activator inhibitor-1 in the biological sample.

In a related embodiment, a first detection reagent such as avidin or biotin is immobilized on the insoluble support. An antibody to PAI-1 binding molecule is then contacted with the immobilized detection reagent. The antibody can recognize PAI-1 binding molecule upon contact. The sample is then contacted with PAI-1 binding molecule bound to an antibody, which is bound to a detection reagent immobilized on the insoluble support. In a washing step unbound components are removed from insoluble support. The insoluble support is contacted with anti-plasminogen activator-1 inhibitor antibody to form antibody-antigen complexes. The unbound antibody from insoluble support is removed and the antibody-antigen complexes are detected using a second detection reagent. The amount of antibody-antigen complex in the biological sample is determined and the measured amounts are correlated with a standard curve (e.g., a standard curve established using a STABLY ACTIVE PAI-1). The results are indicative of the amount of active plasminogen activator inhibitor-1 in the biological sample.

In some embodiments, the insoluble support can be modified in a manner that would allow tethering of the PAI-1 binding molecules to the insoluble support. Tethering can include, without limitation, binding a chemically modified PAI-1 binding molecule to an insoluble surface (support) with matching chemistry by methods well-known in the art such as binding a biotin-labeled proteinase to avidin-coated plates or the reverse, covalent attachment of the proteinase through sulfhydryl reactive insoluble supports, binding of 6-X His-tagged proteinases to metal impregnated insoluble supports, and/or binding of a serine proteinase through a specific tag to a receptor coated onto the surface that recognizes and binds the tag. Any surface modification that enhances the overall protein-binding properties of the plastic polymer may be used as an insoluble support to introduce different functional groups onto a polystyrene surface. Descriptions of these methodologies are well known in the art and are described, e.g., Butler, “The behavior of antigens and antibodies immobilized on a solid phase” (MHV Van Regenmortel, ed. Structure of Antigens, Vol. 1, pp. 209-59, 1992, CRC Press, Boca Raton, Fla.).

In some embodiments, the PAI-1 binding molecule can be added directly to the sample to be analyzed without first having been bound to the insoluble support, but can then be captured by the insoluble support through a specific process including, but not limited to, those already described herein. For example, it is well known that proteins will adsorb to an insoluble surface such as polystyrene. Many studies have also indicated that surface modifications such as the introduction of different functional groups onto the polystyrene surface will enhance the overall protein-binding properties of the plastic polymer (Butler. The behavior of antigens and antibodies immobilized on an insoluble phase. In: Van Regenmortel MHV, ed. Structure of Antigens, Vol. 1. Boca Raton, Fla.: RC Press, 1992:209-59).

In addition, proteins may be modified (e.g. via a specific tag or mutation), and the modified protein used to detect the protein of interest by direct addition to the sample, followed by a solution reaction, and then detection using an insoluble support system such as those described herein.

Further, the present invention with alterations in volumes, can be easily adapted to other support systems such as a dipstick. For example, a dipstick assay can involve collection of a fluid (e.g., urine) into which the sticks are placed for a fixed period of time and then removed. Such assays typically involve a single step, however may have additional steps to facilitate detection. The dipsticks are generally read manually but can also be placed in instruments for semi-quantitative analysis. Lateral flow devices follow the same principle (capillary action), but are generally housed in a cassette casing (e.g., a plastic casing) and in this instance a drop of biological fluid (blood, urine, plasma, serum, or saliva) is tested. These devices use less sample, but can be more qualitative. The test strips can use a small drop of fluid and can be read by an instrument. For example, a dipstick, test strip, or lateral flow device containing a binding strip impregnated with a capture molecule (e.g., an enzyme, antibody, biotin, or avidin) by the methods known in the art can be used. In some cases a dried detection antibody coupled to colloidal gold particles is used to detect bound antigen. If the sample is whole blood, the device generally contains a filter to remove cells prior to entry into the device. The plasma of the sample then wicks through the device by capillary action. Active PAI-1 binds to the capture molecule and is quantitated by visualization of the gold particles, which form a band or a series of bands depending on the exact way the device is designed. The terms “dipstick” “test strips,” and “lateral flow” assays are used interchangeably herein.

In yet other embodiments, the present invention provides kits, which can be employed in the assay. The kits include one or more of the following components in an amount sufficient to perform at least one assay: a composition containing an anti-PAI-1 polyclonal or monoclonal antibody or fragments thereof; as a separately packaged reagent, PAI-1 standards consisting of STABLY ACTIVE PAI-1; uPA or tPA coated insoluble support matrix such as strips, dipsticks, microbeads, or microtiter plates; and buffers. Instructions for use of the packaged reagent are also typically included. “Instructions for use” typically include a tangible expression describing the reagent concentration or at least one assay method parameter such as the relative amounts of reagent and sample to be admixed, maintenance time periods for reagent/sample admixtures, temperature, buffer conditions and the like. Also included, in one form or another, may be charts, graphs and the like that demonstrate predetermined concentration levels correlating specific physiological conditions to PAI-1 events. Microtiter plates can be replaced with other support systems such as a dipstick or micro-beads. The former are useful, e.g., for rapid semi-quantitative analysis, and the latter are useful, e.g., in an automated assay format. An automated assay may provide quantitative results comparable to a microtiter plate analysis.

In yet other embodiments, the present invention provides a diagnostic assay for identifying a subject (e.g., patient) at risk for PAI-1 related disorders, and therefore for identifying suitable patients for therapy or for monitoring a subject during therapy. The novel diagnostic assay described herein provides a method for monitoring patients that are being treated for PAI-1 related disorders, e.g., post-myocardial infarction, cancer, and Type 2 diabetes. Furthermore, accurate measurement of plasma PAI-1 can be useful in the development of therapeutic agents that can restore endogenous stimulation of fibrinolysis through PAI-1 inhibition, e.g., by providing a sensitive method of determining the efficiency of a therapeutic agent.

Any therapeutic agent that will restore endogenous stimulation of fibrinolysis through PAI-1 inhibition must be developed through accurate measurement of plasma PAI-1, and this cannot be readily accomplished with current diagnostic systems. The present invention represents a novel method for the accurate determination of PAI-1 using a novel technique. Accordingly, the present invention provides a method of screening agents (compounds) to identify those that can enhance or inhibit active PAI-1.

The practice of the present invention employs, unless indicated specifically to the contrary, known methods of virology, immunology, microbiology, molecular biology, and recombinant DNA techniques within the skill of those in the art. Some of the methods are described below for the purpose of illustration. Such techniques are explained fully in the literature. See, e.g., Sambrook, et al., Molecular Cloning: A Laboratory Manual (2nd Edition, 1989); Maniatis et al., Molecular Cloning: A Laboratory Manual (1982); DNA Cloning: A Practical Approach, vol. I & II (Glover and Hames, eds. Oxford University Press, 1995); Oligonucleotide Synthesis (Gait, ed., Oxford University Press, 1984); Nucleic Acid Hybridization: A Practical Approach (Hames and Higgins, eds., Oxford University Press, 1990); Transcription and Translation: A Practical Approach (Hames and Higgins, eds., IRL Press, Oxford, 1984); Freshney's Culture of Animal Cells, John Wiley and Sons, Inc., 1998); Perbal, A Practical Guide to Molecular Cloning, 2^{nd} Edition, John Wiley and Sons, Freshney, (1988).

All publications, patents and patent applications cited herein, whether supra or infra, are hereby incorporated by reference in their entirety.

Abbreviations and Definitions:

The following definitions are provided for the full understanding of terms and abbreviations used in this specification.

As used herein and in the appended claims, the singular forms “a”, “an”, and “the” include the plural reference unless the context clearly indicates otherwise. Thus, for example, a reference to “an antibody” includes a plurality of such antibodies, and a reference to “an inhibitor” is a reference to one or more inhibitors and equivalents thereof known to those skilled in the art, and so forth.

The abbreviations in the specification correspond to units of measure, techniques, properties or compounds as follows: “Sec” means second(s), “min” means minutes, “h” means hour(s), “d” means day(s), “kg” means kilogram(s), “g” means gram(s), “mg” means milligram(s), “μg” means microgram(s), “ng” means nanogram(s), “kDa” means kilodalton(s), “°C.” means degree(s) Celsius, “cm” means centimeter(s), “μL” means microliter(s), “mL” means milliliter(s), “mM” means millimolar, “M” means molar, “mmole” means millimole(s), “ng/ml ” means nanogram per milliliter, and “U” means Units.

“Sodium dodecyl sulfate” is abbreviated SDS.

“Polyacrylamide gel electrophoresis” is abbreviated PAGE.

“Sodium dodecyl sulfate-polyacrylamide gel electrophoresis” is abbreviated SDS-PAGE.

“Enzyme linked immunosorbent assay” is abbreviated ELISA

“N-[2-hydroxyethyl]piperazine-N′-[2-ethanesulfonic acid]” is abbreviated HEPES.

“Isopropyl-beta-D-thiogalactopyranoside” is abbreviated IPTG.

“plasminogen activator inhibitor” is abbreviated PAI.

“plasminogen activator” is abbreviated PA.

“Tissue-plasminogen activator” is abbreviated tPA.

“Single chain tPA is abbreviated sc tPA.

“Two chain tPA” is abbreviated tc tPA.

“Urokinase type plasminogen activator” is abbreviated uPA.

“Single chain uPA is abbreviated sc uPA.

“Two chain uPA” is abbreviated tc uPA.

“National Institute for Biological Standards and Control” is abbreviated NIBSC.

In the context of this disclosure, a number of terms shall be utilized. As used herein, the term “plasminogen activator inhibitor” (PAI) is meant to indicate a protein that inhibits or checks the action of a plasminogen activator.

In general, “Plasminogen activator inhibitor” or “PAI-1”, as well as PAI-1-related polypeptides, refers, without limitation, to a substance that inhibits the action of plasminogen activator. The term “PAI-1” also refers to, without limitation, polypeptides having the amino acid sequence as described in Pannekoek et al. (EMBO J. 5(10):2539-2544 (1986)), Gils et al. (Biochim. Biophys. Acta. 387(1-2):291 -297 (1998)); Sui et al. (Biochem. J. 331 (Pt 2):409-415 (1998)); Ginsburg. et al. (J. Clin. Invest., 78:1673-1680 (1986)), or those described in U.S. Pat. Nos. 6,303,338; 6,103,498, as well as wild-type PAI-1 derived from other non-human species, such as, e.g., bovine, porcine, canine, murine, and rat PAI-1. In practicing the present invention, any PAI-1 polypeptide may be used that interacts with plasminogen activator. This includes PAI-1 polypeptides derived from blood or plasma, or produced by recombinant means. Accordingly, the term “PAI-1” is intended to encompass all naturally occurring PAI-1 polypeptides in both active including constitutively active forms, e.g., PAI-1 (14-1 b, Molecular Innovations, Southfield Mich. and latent conformations.

PAI-1 is a principal physiological inhibitor of both forms of plasminogen activators (PAs), uPA (urokinase-type plasminogen activator) and tPA (tissue-type plasminogen activator). PAI-1 is secreted in an active form, which spontaneously converts to an inactive latent form. It can also be partially stabilized in the active form by binding to the plasma protein vitronectin.

The present invention further encompasses natural allelic variations of PAI-1 that may exist and occur from one individual to another. Also, degree and location of glycosylation or other post-translation modifications may vary depending on the chosen host cells and/or the nature of the host cellular environment. The term “PAI-1 ” is also refers to PAI-1 polypeptides in their zymogen form, as well as those that have been processed to yield their respective active forms. The term “PAI-1 -related polypeptides” include such polypeptides in their zymogen form, as well as those that have been processed to yield their respective active forms.

“PAI-1-related polypeptides” also include, without limitation, polypeptides exhibiting substantially the same or improved biological activity relative to wild-type human PAI-1, polypeptides, in which the PAI-1 biological activity has been substantially modified or reduced relative to the activity of wild-type human PAI-1, and/or contain one or more amino acid sequence alterations relative to human PAI-1 (i.e., PAI-1 variants), and/or contain truncated amino acid sequences relative to human PAI-1 (i.e., PAI-1 fragments). Such PAI-1-related polypeptides may exhibit different properties relative to human PAI-1, including stability, phospholipid binding, altered specific activity, and the like. These polypeptides include, without limitation, PAI-1 that has been chemically modified and PAI-1 variants into which specific amino acid sequence alterations have been introduced that modify or disrupt the activity of the polypeptide.

PAI-1 -related polypeptides, further include variants of PAI-1, whether exhibiting substantially the same or better activity than wild-type PAI-1, or, alternatively, exhibiting substantially modified or reduced activity relative to wild-type PAI-1, include, without limitation, polypeptides having an amino acid sequence that differs from the sequence of wild-type PAI-1 by insertion, deletion, or substitution of one or more amino acids.

The present invention includes the use of PAI-1 polypeptides and its fragments thereof, such as, e.g., those having the amino acid sequence disclosed in Gils et al. (Biochim, Biophys, Acta. 1387(1-2):291-7 (1998)), Sui et al. (Biochem. J.; 331 (Pt 2):409-15 (1998)), Ginsburg et al. (J. Clin. Invest., 78:1673-1680 (1986)), those described in U.S. Pat. Nos. 6,303,338; 6,103,498, or as disclosed in Pannekoek et al. (EMBO J. 5(10):2539-2544 (1986)) (wild-type PAI-1). The invention further encompasses, without limitation, use of “mutant PAI-1” (e.g. “STABLY ACTIVE PAI-1”), polypeptides, such as, e.g., those having the amino acid sequence disclosed in Berkenpas et al. (EMBO J. 14:2969-2977, (1995)) and U.S. Pat. No. 6,103,498.

“Active PAI-1” refers to those fragments, derivatives and analogs of PAI-1 polypeptide displaying one or more known functional activities associated with a full-length (wild-type) active PAI-1 polypeptide (e.g., inhibiting PA, binding to an anti-PAI-1 antibody, and the like. “STABLY ACTIVE” refers to an active PAI-1 that stays active throughout the experiment. STABLY ACTIVE PAI-1 may be (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more of the amino acid residues includes a substituent group, or (iii) one in which the mature polypeptide is fused with another compound, such as a compound to increase the half-life of the polypeptide (for example, polyethylene glycol), or (iv) one in which the additional amino acids are fused to the mature polypeptide such as a leader or secretory sequence or a sequence which is employed for purification of the mature polypeptide or a precursor protein sequence. A STABLY ACTIVE PAI-1 may also be a naturally occurring variant such as a naturally occurring allelic variant, or it may be a variant that is not known to occur naturally. Among STABLY ACTIVE PAI-1's in this regard are polypeptides that differ from the aforementioned polypeptides by amino acid substitutions, deletions or additions. The substitutions, deletions or additions may involve one or more amino acids. Alterations in the sequence of the amino acids may be conservative or non-conservative amino acid substitutions, deletions or additions. All such polypeptides defined above are deemed to be within the scope of those skilled in the art from the teachings herein and from the art.

In general, useful amino acid substitutions or mutations are those that significantly increase PAI-1 structural and/or functional stability. Functional stability (half-lives or T1/2) of these mutants would be exceeding that of wild-type PAI-1 by more than about one-fold, e.g., more than about two-fold, more than about five-fold, or higher. Examples of such amino acid substitutions include K154T, Q319L, M354I, N150H and 191 L or combinations thereof. More examples of PAI-1 mutants that are STABLY ACTIVE are described in Berkenpas et al. (EMBO J. 14:2969-2977 (1995)).

A stably active human PAI-1 referred to herein as STABLY ACTIVE PAI-1 is used to prepare standard curves of the present invention. Various ranges of the STABLY ACTIVE PAI-1 concentrations are prepared. The amount of active plasminogen activator inhibitor-1 in the sample can be determined by detecting the amount of active PAI-1 complex and correlating this amount of complex to the amount of active PAI-1 from a standard curve, which curve is generated, under the same conditions, but replacing biological sample with assay diluent. An active PAI-1 complex is then formed comprising PAI-1 binding molecule and STABLY ACTIVE plasminogen activator inhibitor-1.

The term “assay diluent” refers to a solution that dilutes the assay samples prior to assay. While most assay buffers can be used (See, e.g., Current Protocols in Immunology Wiley/Greene, N.Y.; Harlow and Lane (1989); Antibodies: A Laboratory Manual, Cold Spring Harbor Press, N.Y.; Stites et al. (eds.) Basic and Clinical Immunoloay (4TH ed.) Lange Medical Publications, Los Altos, Calif., and references cited therein), particularly preferred assay diluents comprise buffering salts, including water, saline, Tris, carbonate, phosphate, borate, citrate, HEPES, etc.; sodium or another alkali salt and a preservative to prevent microbial growth. Particularly preferred assay diluents have an effective buffering capacity of between about pH 7 to about pH 9 (in the range of the biological sample to be tested. In a preferred embodiment, the assay diluent is selected from biological fluids that are stripped off endogenous human PAI-1, i.e. as PAI-1 depleted plasma or PAI-1 deficient plasma, e.g., PAI-1 immuno/activity-depleted plasma.

To prepare PAI-1 -depleted plasma, purified anti-PAI-1 antibodies can be immobilized on CNBr-activated Sepharose™ following the manufacturer's instructions. Plasma can be depleted of PAI-1 by passing over the immobilized antibody column. All PAI-1 antigen is removed from the plasma as assessed by two different EIAS, i.e., TINTELIZA PAI-1 (Biopool; Umea, Sweden; catalog number 210221) and INNOTEST PAI-1 (Innogenetics BA, Antwerp, Belgium).

The PAI useful herein can be from a number of sources such as human endothelial cells, placental extracts, platelets, plasma and serum, a transformed or neoplastic cell line (e.g., HT 1080), or that proteinaceous molecule prepared by recombinant techniques such as a fusion polypeptide as described herein and is known in the art. It can also be from other mammalian sources such as bovine aortic endothelial cells (BAEs) and CHO cell lines. Other recombinant hosts would include prokaryotic cell lines such as E. coli, phage, insect cell lines and baculovirus.

“PAI-1 binding molecule” refers to protein or non-protein molecules which bind or interact with PAI-1 or variants or fragments thereof, for example enzymes, cell components, polypeptides, peptides, antibodies and antibody-derived reagents, nucleic acid molecules, RNA molecules, and small molecules. Examples of PAI-1 binding molecule include but are not limited to serine proteases, tPA, uPA, vitronectin, glycosaminoglycan, fibronectin, cathepsin G and prostate specific antigen and combinations thereof. Examples of serine proteases include but are not limited to chymotrypsin, neutrophil elastase, pancreatic elastase, trypsin, plasmin, thrombin, acrosomal protease, complement C1, keratinase, collagenase, fibrinolysin, cocoonase, and combinations thereof.

“Plasminogen activator” is a protein that activates plasminogen present in blood, particularly in plasma, and converts it into plasmin in the fibrinolytic system of blood clotting. Plasminogen activators useful in the present invention include tissue-type plasminogen activator (tPA) and urokinase-type plasminogen activator (u-PA), their variants and fragments thereof. There are several plasminogen activators (PA) including, but not limited to, tissue-type PA (tPA: including single chain tPA and two chain tPA), urokinase PA (u-PA: including the proenzyme form referred to as prourokinase, or single chain urokinase PA (scuPA); high molecular weight two chain uPA and low molecular weight uPA), and streptokinase, which are capable of converting inactive zymogen plasminogen to the active enzyme, plasmin.

As used herein, “urokinase-type” is meant to indicate urokinase and its homologous proteins as found in mammals other than humans.

“PAI-1 related disorder” refers to a disorder characterized with altered PAI-1 level or activity. PAI-1 related disorders include, but are not limited to, thromboembolic disease, inherited autosomal recessive bleeding disorder, thrombosis or fibrinolytic impairment in a mammal associated with formation of atherosclerotic plaques, venous and arterial thrombosis, myocardial ischemia, atrial fibrillation, deep vein thrombosis, coagulation syndromes, pulmonary fibrosis, cerebral thrombosis, thromboembolic complications of surgery or peripheral arterial occlusion diseases associated with extracellular matrix accumulation (e.g., renal fibrosis, chronic obstructive pulmonary disease, polycystic ovary syndrome, restenosis, renovascular disease and organ transplant rejection), malignancies and diseases associated with neoangiogenesis (e.g., diabetic retinopathy), cancer (e.g., breast and ovarian cancer), inflammatory disease, septic shock, vascular damage associated with infection, Alzheimer's disease, myelofibrosis, diabetic nephropathy, renal dialysis associated with nephropathy, septicemia, obesity, insulin resistance, proliferative diseases (e.g. psoriasis), cerebrovascular disease, microvascular disease (e.g., nephropathy, neuropathy, retinopathy, and nephrotic syndrome), hypertension, diabetes and related diseases, hyperglycemia, hyperinsulinemia, malignant lesions, premalignant lesions, gastrointestinal malignancies, liposarcomas tumor and epithelial tumor, dementia, osteoporosis, arthritis, asthma, heart failure, arrhythmia, angina, atherosclerosis, osteopenia, low grade vascular inflammation, stroke, coronary heart disease, myocardial infarction, peripheral vascular disease, peripheral arterial disease, acute vascular syndromes and wound healing, and scarring.

The term “immunogen”, as used herein, describes an entity that induces antibody production in the host animal. In some instances, the antigen and immunogen are the same entity, while in other instances, the two entities are different. Immunogens used to elicit antibodies of this invention include, but are not limited to, PAI-1/tPA complexes and STABLY ACTIVE PAI-1 or wild type human or other mammalian species of PAI-1's or PAI-1 in complex with other ligands such as vitronectin, polyanionic substances such as heparin, dextran sulfate, dermatan sulfate, and DNA. Such antibodies may be generated using standard techniques described herein, against the PAI-1 itself or against peptides corresponding to portions of the protein. Such antibodies include but are not limited to polyclonal, monoclonal, Fab fragments, single chain antibodies, or chimeric antibodies.

As used herein, the terms “label” and “indicating means” in their various grammatical forms refer to atoms or molecules that are either directly or indirectly involved in the production of a detectable signal to indicate the presence of a complex. Any label or indicating means can be linked to or incorporated in an antibody molecule that is part of an antibody or monoclonal antibody composition or any other PAI-1 binding molecule of the present invention, or used separately, and those atoms or molecules can be used alone or in conjunction with additional reagents. Such labels are themselves well known in clinical diagnostic chemistry and constitute a part of this invention insofar as they are utilized with otherwise novel methods and/or systems.

These labeling means comprise but are not limited to markers selected from the group consisting of radioisotopes, enzymes, chemical or chemico-luminescent markers, biotin, electron dense molecules, fluorochromes haptens, antibodies and any other detectable label. Further, there are many labels and methods of labeling known in the analytical art that are useful in the present invention. Those of ordinary skill in the art will know of other suitable labels and methods of conjugating these labels to a reagent or conjugating a molecule to the reagent that generates a label, or is capable of ascertaining such methods using routine experimentation.

Detection can be facilitated by coupling the antibody to a detectable substance. Examples of detectable substances include various enzymes, prosthetic groups, fluorescent materials, luminescent materials, bioluminescent materials, radioactive materials, positron emitting metals using various positron emission tomographies, and nonradioactive paramagnetic metal ions. The detectable substance may be coupled or conjugated either directly to the antibody (or fragment thereof) or indirectly, through an intermediate (such as, for example, a linker known in the art) using techniques known in the art (for example, U.S. Pat. No. 4,741,900 for metal ions that can be conjugated to antibodies for use as diagnostics in the present invention). Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include 125I, 131I, 111In or 99Tc.

The term “fluorescent labeling” means the antibody is made fluorescent by coupling or forming a complex with a suitable fluorescent agent such as fluorescein iso(thio)cyanate. Suitable fluorescent labeling agents include but are not limited to fluorochromes such as fluorescein isocyanate (FIC), fluorescein isothiocyanate (FITC), 5-dimethylamine-1-naphthalenesulfonyl chloride (DANSC), lissamine, rhodamine 8200 sulphonyl chloride, and tetramethylrhodamine isothiocyanate (TRITC) (RB200 SC. A description of immunofluorescence analysis techniques is found in DeLuca “Immunofluorescence Analysis”, in Antibody As A Tool, Marchalonis et al., eds., John Wiley & Sons, Ltd., pp. 189-231 (1982), which is incorporated herein by reference.

The term “radioactive labeling” means the antibody carries a radioactive isotope allowing the assay to be carried out by a radioactivity count, the isotope being carried either on one element of the antibody structure, for example constitutive tyrosine residues, or on an appropriate radical attached to it. The term “enzymatic labeling” means the specific antibody is coupled to an enzyme which, combined with the use of suitable reagents, allows the specific antibody to be quantitatively measured. When a fluorescent antibody is used, the fluorescence of the sample tested is read directly on a suitable apparatus. When an enzyme attached to a specific antibody is used, the stained or fluorescent product is obtained by adding a solution containing the enzyme substrate and one or more additional agents which result in a final product that is either a stained product soluble in the medium, an insoluble stained product or a soluble fluorescent product, as explained above. Next, the light signal is measured using a device adapted to each situation: transmission photometer, reflection photometer or fluorometer.

In some cases, assays as described herein are conducted in which active PAI-1 in a sample is bound to a PAI-1 binding molecule (e.g., a plasminogen activator) and the interaction between the two molecules is detected, e.g., using fluorescence energy transfer (FET) (for example, Lakowicz et al., U.S. Pat. No. 5,631,169; Stavrianopoulos et al., U.S. Pat. No. 4,868,103; and fretimaging.org/mcnamaraintro.html). A fluorophore label on the first, ‘donor’ molecule is selected such that the donor's emitted fluorescent energy is absorbed by a fluorescent label on a second, ‘acceptor’ molecule, which in turn fluoresces due to the absorbed energy. Alternately, the ‘donor’ protein molecule can utilize the natural fluorescent energy of tryptophan residues. Labels are chosen that emit different wavelengths of light, such that the ‘acceptor’ molecule label can be differentiated from that of the ‘donor’. Since the efficiency of energy transfer between the labels is related to the distance separating the molecules, the spatial relationship between the molecules can also be assessed. In a situation in which binding occurs between the molecules, the fluorescent emission of the ‘acceptor’ molecule label in the assay should be maximal. An FET binding event can be conveniently measured using known fluorometric detection methods that are known in the art (e.g., using a fluorimeter).

In another example of a method for detecting the interaction between two molecules in an assay described herein, determining the binding between a PAI-1 and a PAI-1 binding molecules (e.g., a plasminogen activator) can be accomplished using real-time Biomolecular Interaction Analysis (BIA) (e.g., Sjolander and Urbaniczky, 1991, Anal. Chem. 63:2338-2345 and Szabo et al., 1995, Curr. Opin. Struct. Biol. 5:699-705). “Surface plasmon resonance” or “BIA” detects biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal that can be used as an indication of real-time reactions between biological molecules.

The terms ” insoluble phase support” or “insoluble support” mean any support capable of binding molecules such as antibodies, nucleic acid fragments, proteins, peptides, polypeptides and combinations thereof. Well-known supports, or carriers, include, but are not limited to, polyethylene, polystyrene, substituted polystyrene, e.g., aminated or carboxylated polystyrene, polyacrylamides, polyamides, polyvinylchoride; magnetic particles, agarose polypropylene, dextran, nylon, glass, amylases, natural and modified celluloses, agaroses, polyacrylamides, magnetite or combinations thereof. The support material may have virtually any structural configuration that is capable of binding biomolecules (i.e., capable of binding to an enzyme that can bind to the PAI-1 target). An “insoluble phase support” can be microtiter wells, tubes or dipsticks, and the like. The support configuration can be spherical, as in a bead, or cylindrical, as in the inside surface of a test tube, or the external surface of a rod. Alternatively, the surface may be flat such as a sheet, test strip, and the like. Suitable carriers are well known to those skilled in the art and can be at the bottom and sides of a polystyrene microtiter plate well.

“Biological sample” refers to a sample of tissue or fluid isolated from a subject, including but not limited to, for example, blood, plasma, serum, fecal matter, urine, bone marrow, bile, spinal fluid, lymph fluid, samples of the skin, external secretions of the skin, respiratory, intestinal, and genitourinary tracts, tears, saliva, milk, blood cells, organs, biopsies and also samples of in vitro cell culture constituents including but not limited to conditioned media resulting from the growth of cells and tissues in culture medium, e.g., recombinant cells, and cell components.

“Body fluid” or “biological fluid” refers to any body fluid including, without limitation, serum, plasma, lymph fluid, synovial fluid, follicular fluid, seminal fluid, amniotic fluid, milk, whole blood, sweat, urine, cerebrospinal fluid, saliva, sputum, tears, perspiration, mucus, tissue culture medium, tissue extracts, and cellular extracts. It may also apply to fractions and dilutions of body fluids. The source of a body fluid can be a human, animal body, an experimental animal, a plant, or other organism.

An assay can be conducted at temperature that permits the reaction between a PA and PAI-1. The temperatures at which the sample is prepared and PAI-1 is measured may be the same or different. For example, the temperature(s) are less than that at which protein precipitates in the sample (about 42° C.) and greater than about 10° C. It can be convenient to prepare the sample at about room temperature. A suitable temperature for use with a microplate reader to measure PAI-1 amount, such as described below, is about 25° C. In some cases, incubations are carried out at a physiological temperature.

The invention also encompasses a method of identifying an agent that can modulate active PAI-1. The method is carried out by performing an assay for PAI-1 as described herein (e.g., using a STABLY ACTIVE PAI-1 to establish a standard curve) in which the amount of active PAI-1 in a sample is determined in the presence and in the absence of a test agent. A test agent that modulates (i.e., increases or decreases) the amount of active PAI-1 in a sample is useful for modulating (i.e., increasing or decreasing active PAI-1. A test agent can be selected from among molecules known in the art, including, without limitation, peptides, peptidomimetics (e.g., peptoids), nucleic acid molecules (e.g., oligonucleotides, siRNAs, antisense RNAs, and ribozymes), small non-nucleic acid organic molecules, and small inorganic molecules. Such molecules can be e.g., designed to bind (e.g., specifically bind) to an active PAI-1 molecule, or can be obtained from a chemical library. In some embodiments, multiple test agents are used in a single initial assay. If it appears that at least one test agent used in the assay modulates active PAI-1, then the individual test agents used in the initial assay are tested individually for their ability to modulate PAI-1, thereby identifying the test agent or test agents in the initial assay that have activity.

Non-limiting examples of sources for test agents include, e.g., combinatorial library made using methods known in the art, including: biological libraries; peptoid libraries (libraries of molecules having the functionalities of peptides, but with a novel, non-peptide backbone which are resistant to enzymatic degradation but which nevertheless remain bioactive (see, e.g., Zuckermann et al., 1994, J. Med. Chem. 37:2678-85); spatially addressable parallel solid phase or solution phase libraries; synthetic library methods requiring deconvolution; the ‘one-bead one-compound’ library method; and synthetic library methods using affinity chromatography selection. Compounds that can be used as test agents are also available from commercial sources (e.g., Leadgenix; Taejon, Korea and Mimotopes; San Diego, CA). Nucleic acids can be designed and synthesized using methods known in the art, and can be obtained, e.g., from commercial services (e.g., Dharmacon, Layfayette, Colo.).

### EXAMPLES

The present invention is further defined in the following Examples, in which all parts and percentages are by weight and degrees are Celsius, unless otherwise stated. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. It is, therefore, intended that the appended claims cover all such equivalent variations as fall within the true spirit and scope of the invention.

### Example 1

General Methods

Preparation of PAI-1 Immuno/Activity Depleted Plasma

50-100 ml of human citrated plasma containing 0.02% sodium azide is passed over a column of immobilized monoclonal antibodies to human PAI-1. The affinity resin consists of approximately 2.0 ml of resin with 2 mg each of the following two antibodies; 6712F2 and 6712A7 (Molecular Innovations Inc, Southfield, Mich.). The affinity resin used to couple the antibodies was Affi-Gel 10 (Biorad Inc., Richmond, Calif.). The manufacturer's instructions were followed for the coupling. The column is pre-equilibrated in a sodium phosphate buffer (50 mM sodium phosphate; 0.1M NaCl pH 7.4). The first 10 ml of plasma is allowed to pass through and is discarded. The flow-through is then recirculated through the column three more times to remove PAI-1 antigen. To insure that all PAI-1 activity is removed the plasma is then incubated at 37° C. for 72 hours to thermally inactivate any traces of PAI-1 activity which may remain.

Preparation of High Molecular Weight Two Chain Urokinase PA (UPA)

To prepare a two chain UPA, a pharmaceutical preparation, Rheotromb® (Curasan AG, Kleinostheim, Germany) was used as the starting material. The pharmaceutical preparation is supplied as an injectable form with Dextran 40 as an added carrier. The material was processed for use in an ELISA (enzyme-linked immunosorbent assay) based application described as follows: five vials of 500,000 IU each were dissolved in 3 ml of the following buffer: 0.05 M sodium phosphate; 0.1 M NaCl; 1 mM EDTA; pH 6.6. The sample was then applied to a Sephacryl™ S-200 (Pharmacia, Piscataway, N.J.) size exclusion resin equilibrated in the application buffer using a column with dimensions of 2.5 cm×110 cm (about 540 ml of resin). This gel filtration step removed most of the original Dextran 40 present in present in the original formulation of Rheotromb® and removed very high and very low molecular weight contaminants from the final preparation. Fractions were analyzed by SDS PAGE and pooled. The sample was then concentrated with a stirred filtration apparatus to an appropriate volume and applied to a column of immobilized benzamidine Sepharose™ 4B (Pharmacia Corporation, Piscataway, N.J.). This resin binds the active urokinase allowing any denatured non-reactive material to flow through. It also removes any residual Dextran 40. The active uPA was eluted with a buffer consisting of 0.1 M glycine, 0.15 M NaCl, pH 3.0, and was collected in a buffer (1 M Tris, 0.1 M NaCl, pH 8.0) to neutralize the pH. The urokinase was then concentrated and dialyzed into 0.05 M TRIS-Cl, 0.1 M NaCl, pH 7.4. The concentration was determined by assaying the absorbance at 280 nm. The activity of the preparation was accessed by forming a uPA/PAI-1 complex using a three fold molar excess of PAI-1 and then performing SDS PAGE. uPA forms an SDS stable complex with PAI-1. A preferred preparation of uPA will form about 95% complex with PAI-1 as evidenced by the apparent increase of molecular weight for free uPA of about 54,000 kDa to a molecular weight of about 97,000 (uPA/PAI-1 complex).

Preparation of Single Chain tPA

A pharmaceutical preparation (Actilyse®; Boehringer Ingelheim, Germany) was used as the starting material. The raw material is supplied as an injectable form with arginine phosphate added to increase tPA solubility. The material was processed for use in this ELISA based application by dissolving the tPA in deionized water to a concentration of approximately 3 mg/ml and then dialyzing the tPA into 0.5 M HEPES: 0.5 M NaCl; pH 7.4. The high concentration of HEPES keeps the tPA soluble while removing arginine, which might otherwise interfere with binding of tPA to ELISA plates.

### Example 2

Preparation of Plasminogen Activator Inhibitor

Preparation of the PAI-1 Activity Standard

An E. coli construct containing the stable mutant form of PAI-1 was prepared as described in Berkenpas et al. (EMBO J. 14(13):2969-2977(1995)). E. coli containing the construct were further produced in a 55L commercial fermentation by Waksman Laboratory at Rutgers University in New Jersey using the following medium: Media—Difco LB (BD Diagnostic Systems Sparks, Md.), 10 g/l Difco tryptone, 5 g/l Difco Yeast Extract, and 10 g/l NaCl. The pH was adjusted to 7.2 prior to sterilization and controlled at 7.0 with 5 N NaOH/43.5% H₃PO4. The following protocol was then used. When the cells reached an optical density of 1.0 then the cells were induced with 1 mM IPTG and the induction continued for 2-3 hrs. The cells were centrifuged via a Sharples AS-26VB supercentrifuge, then resuspended in: 50 mM NaPO4 buffer, pH 6.6 with: 10 mM MgCl2, 1 mM EDTA, 1 mM dithiothreitol, 10 μg/ml DNase, 10 μg/ml RNase, 0.2 μg/ml aprotinin, 0.7 μg/ml pepstatin, 0.5 μg/ml leupeptin, 0.05 mM PMSF(phenylmethylsulfonylfluoride). The cells were disrupted using a Manton-Gaulin Homogenizer. The lysate was then centrifuged in a Beckman JS-21 centrifuge for one hour at 6,000 rpm and the pellet resuspended back into the 1 liter of lysis buffer above and frozen until ready for purification.

For purification, the crude lysate was applied to a 600 ml column of heparin Sepharose™ CL-6B (Pharmacia, Piscataway, N.J.) equilibrated in 0.05 M sodium phosphate, 0.1 M NaCl; 1 mM EDTA; pH 6.6. The column was then washed with the same buffer and the absorbance at 280 nm was monitored until baseline was achieved. The column was washed with several column volumes in the same buffer except without EDTA. The column was then step eluted in the buffer without EDTA but containing 1.0 M NaCl. The PAI-1 containing sample was then applied to a cobalt resin (Talon™, BD Biosciences, Palo Alto, Calif.) in the buffer above without EDTA. The column was then washed to baseline and the PAI-1 eluted with the same buffer containing 300 mM imidazole. The sample was then dialyzed against 0.05 M sodium phosphate; 0.1 M NaCl; 1 mM EDTA pH 6.6 buffer and characterized by SDS PAGE for purity and activity. FIG. 6 is an example of the purity (>99%) of the stably active PAI-1 as imaged by 10% SDS PAGE. Lane 1 shows the purity of the stably active PAI-1, and lane 2 shows the same PAI-1 sample in complex with an excess of human uPA. This image indicates that all of the PAI-1 migrates into complex with the active site containing chain of urokinase with less than 5% of the stably active mutant forming a nonproductive, cleaved byproduct which is a normal process of the bifurcation pathway for PAI-1 inhibition (Lawrence et al., J. Biol. Chem. 275:5839 (2000)).

### Example 3

Preparation of Antibodies

The purified primary detection antibody currently used in a PAI-1 assay (MA-33B8, Molecular Innovations Inc.) was diluted to a concentration of 50 μg/ml solution using Poly-Poly conjugate diluent (Immunochemistry Technologies, LLC, Bloomington, Minn.). One ml of the 50 μg/ml solution of primary antibody was then aliquoted into clear glass lyophilization bottles and snap frozen using a dry ice/ethanol bath. The solution was then lyophilized and stored prior to use. The primary antibody was reconstituted with 10 ml of the Poly-Poly diluent making the working primary antibody solution of 5 μg/ml.

A 1:300 dilution of a purified secondary antibody/HRP conjugate (Jackson Immuno Research Laboratories, West Grove, Pa.) was prepared using a conjugate stabilizer (SurModics; Eden Prairie, Minn.). One ml of the 1:300 dilution of secondary antibody was aliquoted into amber glass lyophilization bottles and frozen using a dry ice/ethanol snap freeze process. The solution was lyophilized for storage prior to use. The secondary antibody was reconstituted by using 10 ml of conjugate stabilizer to make a working secondary antibody solution that was a 1:3000 dilution.

Other methods of preparing antibodies are known in the art and those in the art will understand how to select and prepare such antibodies.

### Example 4

Preparation of Assay Plates

High binding polystyrene microtiter wells (Immulon 2 HB; Thermo Labsystems Franklin, Mass.) were coated with uPA (urokinase plasminogen activator) by using a 10 μg/ml solution of uPA made using a universal plate coating buffer (Immunochemistry Technologies, LLC). All wells were coated with 100 μl of the uPA solution; plates were covered with aluminum foil (to protect from light) and allowed to incubate overnight at 25° C. The uPA solution was aspirated from the plate and washed three times using 300 μl of an ELISA wash buffer (Immunochemistry Technologies, LLC, Bloomington, Minn.). After the washing step was completed, 300 μl of a general low level blocker with BSA (Immunochemistry Technologies) was added to all wells and allowed to sit covered with aluminum foil for three hours at 25° C. Blocker was aspirated and plates were lightly covered and allowed to dry overnight.

The plates can be packaged using various methods known in the art. For this example, the plates were packaged using a vapor flex barrier bag purchased from Tiger Pak Corporation (Clifton, N.J.). Dried plates were placed into the bag along with a dust-free desiccant purchased from Engelhard Corporation (Iselin, N.J.). The bags were heat-sealed and labeled. Using the same method for packaging as above, air would then be replaced by an inert gas, such as helium or argon. The inert gas would then be evacuated and the bag heat-sealed and labeled.

### Example 5

Preparation of Standard Curves

A stably active human PAI-1 referred to herein as STABLY ACTIVE PAI-1 was used to prepare standard curves. The STABLY ACTIVE PAI-1 concentrations ranged from 0 to 150 ng/ml. The lyophilized 0 U/ml standard (PAI-1 depleted plasma) and 200 U/ml standard (PAI-1 depleted plasma spiked with STABLY ACTIVE PAI-1) were reconstituted and prepared.

PAI-1 activity standards were prepared from human citrated plasma that had been both immunodepleted and treated for 72 hours at 37° C. degrees in the presence of 0.02% sodium azide to prevent bacterial growth. The PAI-1 sample used for the activity standard (STABLY ACTIVE PAI-1-14-1B stable mutant) was shown to be >99.9% pure by SDS PAGE and formed greater than 98% complex with human uPA. The PAI-1 was diluted to a concentration of 0.1 mg/ml as determined by absorbance at 280 nm and was then serially diluted to spike the plasma to a concentration of 462 ng/ml. Based on a conversion factor of 1.34 ng per tPA Unit PAI-1 activity (Biopool), the activity standards were assigned a value of 345 U/ml. This value was used for the experiments that follow.

### Example 6

Thermal Stability Study Comparing PAI-1 Activity of the STABLY ACTIVE PAI-1 Standard With a PAI-1 NIBSC Activity Standard and a Biopool PAI-1 Activity Standard

The assay method of the present invention was used to compare the activity of the STABLY ACTIVE PAI-1 against an international standard (NIBSC) and a commercially available activity standard (Biopool Chromolyze®) as a function of temperature.

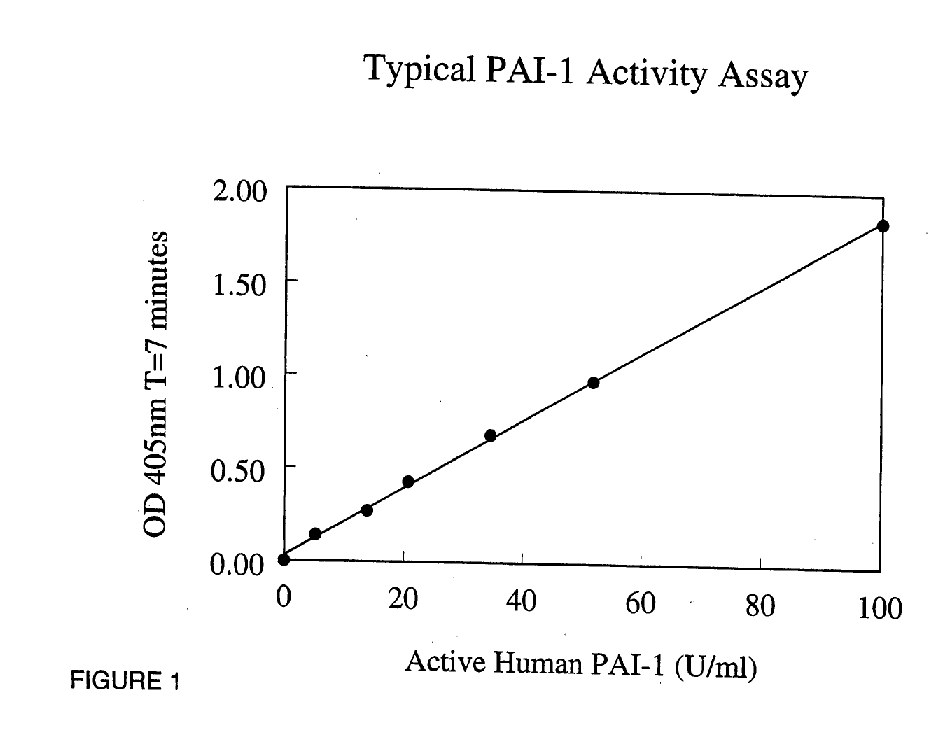

A plate was coated with uPA as described in Example 4. Three sets of samples were prepared for the assay; 1) 80 μL general diluent and 20 μL NIBSC PAI-1 standards in a freshly reconstituted vial of PAI-1 sample, 2) 80 μL general diluent and 20 μL STABLY ACTIVE PAI-1 standard (30 U/ml), and 3) 80 μL general diluent and 20 μL Biopool Chromolize® activity standards (30 U/ml). The activity of each standard in the assay was determined over time at 37° C. PAI-1 activities were measured at zero time and then at each hour for 8 hours. Samples were taken at the indicated time points and snap frozen on dry ice/ethanol for subsequent analysis. These studies were performed twice and the data were averaged. Activity data were calculated by comparison to the single standard curve of FIG. 1.

To conduct the detection portion of the assay, lyophilized primary antibody (MA-33B8) was diluted at 5 μg/ml using 10 ml of poly-poly conjugate diluent (Immunochemistry Technologies). One hundred microliters of the primary antibody was then added to each well and incubated for 30 minutes at 25° C. The wells were again washed three times with wash buffer. A lyophilized secondary antibody (goat anti-mouse horseradish peroxidase conjugate) was diluted at a ratio of 1:3000 using 10 ml StabilZyme® HRP conjugate stabilizer (SurModics, Eden Prairie, Minn.). One hundred microliters of the secondary antibody was added to each well and incubated for 30 minutes at 25° C. The wells were again washed three times with wash buffer. The next step of the assay was to add 100 μL of TMB One substrate (Rainbow Scientific Inc., Windsor, Conn.). The plate was then incubated for five minutes and reaction was quenched by the addition of 50 μl of 1 N sulfuric acid. The absorbance was read at 450 nm and the results were obtained using the standard curve shown in FIG. 1, which was constructed using the STABLY ACTIVE PAI-1 activity standard.

The STABLY ACTIVE PAI-1 standard, unlike the Chromolize® activity standard, had not been standardized against the NIBSC standard. Because the STABLY ACTIVE PAI-1 activity standard has intrinsically more activity than the NIBSC standard, the NIBSC and Chromolize® standard report lower values than would otherwise be the case.

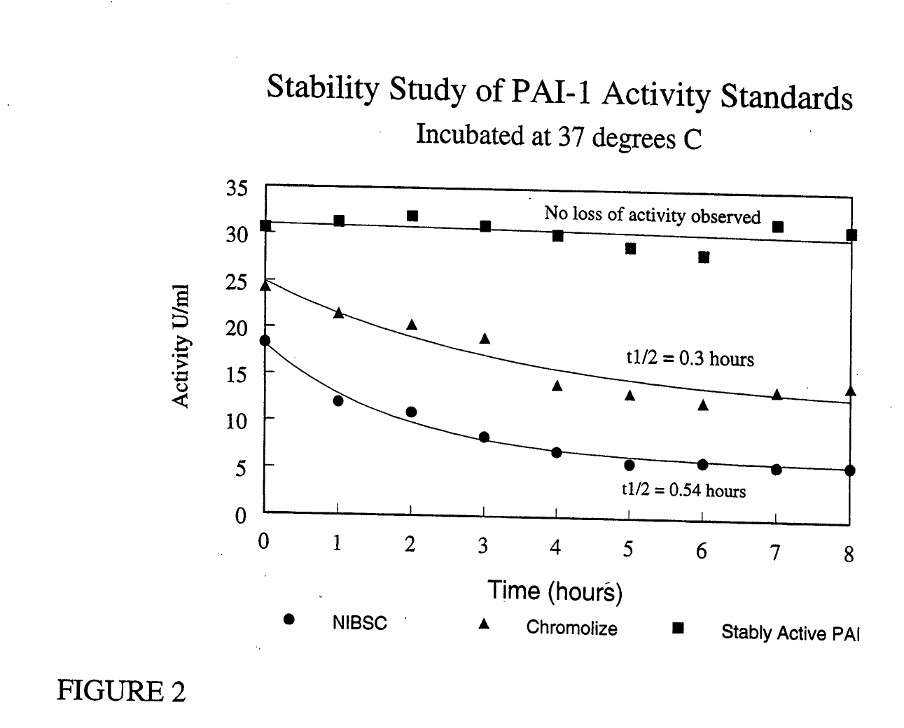

The 30 U/ml STABLY ACTIVE PAI-1 used as a standard (not standardized to the NIBSC activity standard) did not lose any activity over time. However, the Biopool activity standard and the NIBSC activity standard showed substantial losses in activity over the same time course. The data are shown in FIG. 2 and the half-lives are indicated in the figure. In summary, both the Biopool and the NIBSC PAI-1 activity standards are subject to spontaneous loss of activity over time at 37° C., whereas the STABLY ACTIVE standard is refractive to such changes, i.e., has greater thermal stability.

These data demonstrate that the STABLY ACTIVE PAI-1 has improved thermal stability compared to other PAI-1 standards that are in use.

### Example 7

PAI-1 Assay

To perform an assay, high binding polystyrene microtiter wells were coated with tc-uPA (two-chain urokinase plasminogen activator). One hundred microliters of a 10 μg/ml solution of tc-uPA was prepared using a universal plate coating buffer (Immunochemistry Technologies, LLC, Bloomington, Minn.) and was added to the wells. The plate was covered and incubated overnight at 25° C. The solution was aspirated from the plate and washed three times using 300 μl of an ELISA wash buffer (Immunochemistry Technologies). Next, 300 μl of a general low-level blocker with bovine serum albumin (BSA) (Immunochemistry Technologies, LLC, Bloomington, Minn.) was added to all wells, which were covered and incubated at 25° C. for three hours. The blocker was then aspirated, the plates were lightly covered, and were allowed to dry overnight.

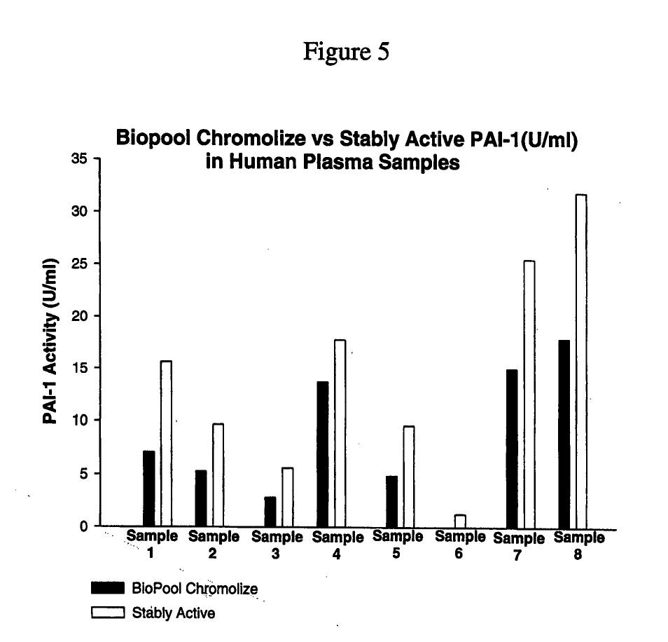

To initiate the assay, 80 μl of General Assay Diluent (Immunochemistry Technologies, LLC, Bloomington, Minn.) was added to all wells of the plate. Twenty microliters of standards or unknown sample (human plasma) was then added to the wells and the plate was incubated for 30 minutes at 25° C. The wells were then washed three times with wash buffer (Immunochemistry Technologies). The lyophilized primary antibody (MA-33B8) was diluted at 5 μg/ml using 10 ml of poly-poly conjugate diluent (Immunochemistry Technologies). One hundred microliters of the primary antibody was then added to each well and allowed to incubate for 30 minutes at 25° C. The wells were again washed three times with wash buffer. The lyophilized secondary antibody (goat anti-mouse horseradish peroxidase conjugate) was diluted at a ratio of 1:3000 using 10 ml StabilZyme® HRP conjugate stabilizer (SurModics, Eden Prairie, Minn.). One hundred microliters of the secondary antibody was added to each well and allowed to incubate for 30 minutes at 25° C. The wells were again washed three times with wash buffer. The next step of the assay was to add 100 μL of TMB One substrate (Rainbow Scientific Inc., Windsor, Conn.). The plate was then allowed to incubate for five minutes and the reaction was quenched by the addition of 50 μl of 1 N sulfuric acid. The absorbance was read at 450 nm and the concentration of PAI-1 in the sample was determined by comparison with the standard curve (FIG. 5).

### Example 8

Comparison of tPA and uPA Coated Plates

The activity of the NIBSC PAI-1 activity standard was calculated using both tc-tPA (Product code #HTPA-TC, Molecular Innovations) coated plates and uPA coated plates. The coating was at 10 μg/ml for each enzyme and performed as described in Example 4. The activity was calculated on the basis of a standard curve constructed using a non-standardized the STABLY ACTIVE PAI-1 standard spiked into depleted plasma as in FIG. 1.

No significant differences were observed in the calculated values for the NIBSC standard (20.8 U/ml using the tPA coated plates and 23.9 U/ml using the uPA coated plates). These results indicate that either tPA or uPA can be used effectively as a capture enzyme in this assay.

### Example 9

Standardization of the 345 U/ml STABLY ACTIVE PAI-1 Standard to the NIBSC PAI-1 Activity Standard

Experiments were conducted to compare the use of STABLY ACTIVE PAI-1 to the NIBSC activity standard. These experiments used immunodepleted plasma that was spiked to contain 345 U/ml based on the STABLY ACTIVE PAI-1 concentration. The PAI-1 concentration was accurately measured by the absorbance at 280 nm, and the purity of the preparation was demonstrated by SDS PAGE. Additionally, the PAI-1 was shown to be fully active based upon complex formation with a molar excess of uPA. In these experiments, SDS PAGE showed that the 43 kDa PAI-1 band completely moved into a 97 kDa band corresponding to the uPA/PAI-1 complex. These experiments provide an example of methods for determining the quality of a PAI-1 (e.g., a STABLY ACTIVE PAI-1) that is used as a standard.

N-terminal analysis or mass spectroscopy can also be routinely performed on lots of the PAI-1 activity standards (e.g., a STABLY ACTIVE PAI-1), if desired, for an independent determination of concentration.

To compare kits and kit components directly, STABLY ACTIVE PAI-1 in plasma was re-standardized to the NIBSC standard. After a series of experiments comparing the STABLY ACTIVE PAI-1 standard to the NIBSC standard, the activity standard (of STABLY ACTIVE PAI-1) was changed from a calculated value of 345 U/ml to 450 U/ml for the experiments that follow.

The NIBSC standard has a reported value of 27.5 U/ml (National Institute for Biological Standards and Control, Herts, UK). After re-standardization, the kit composed by the present inventors using STABLY ACTIVE PAI-1 for the standard curve and the MA-33B8 antibody as the primary antibody resulted in a calculated value of 31 U/ml for the NIBSC standard in experiment (FIG. 3A). The Biopool kit used with all Biopool components reported a calculated value of 26 U/ml for the NIBSC standard (FIG. 3B).

### Example 10

Use of Various Antibodies in PAI-1 Assay

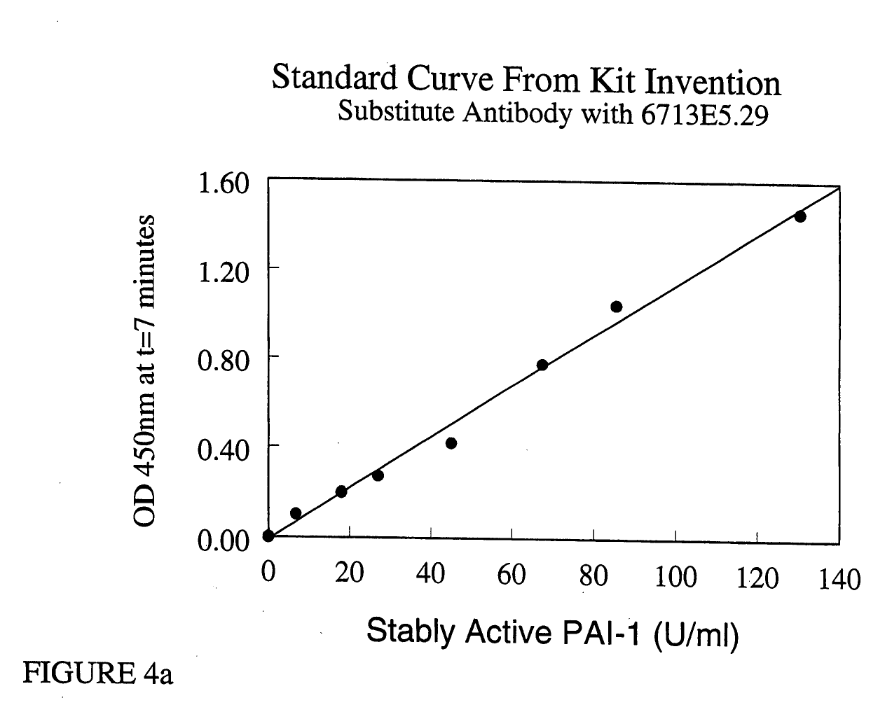

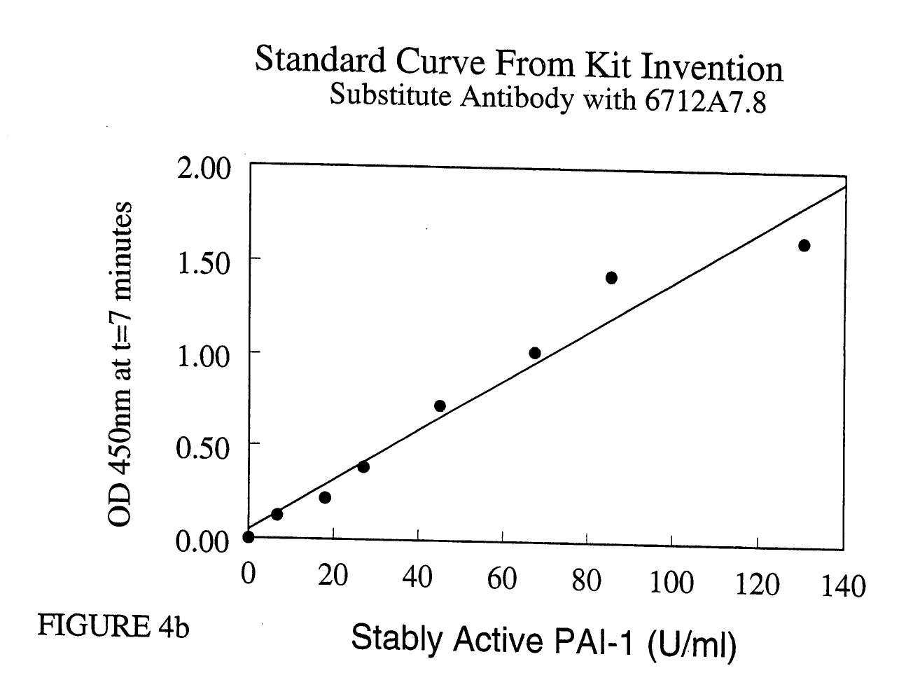

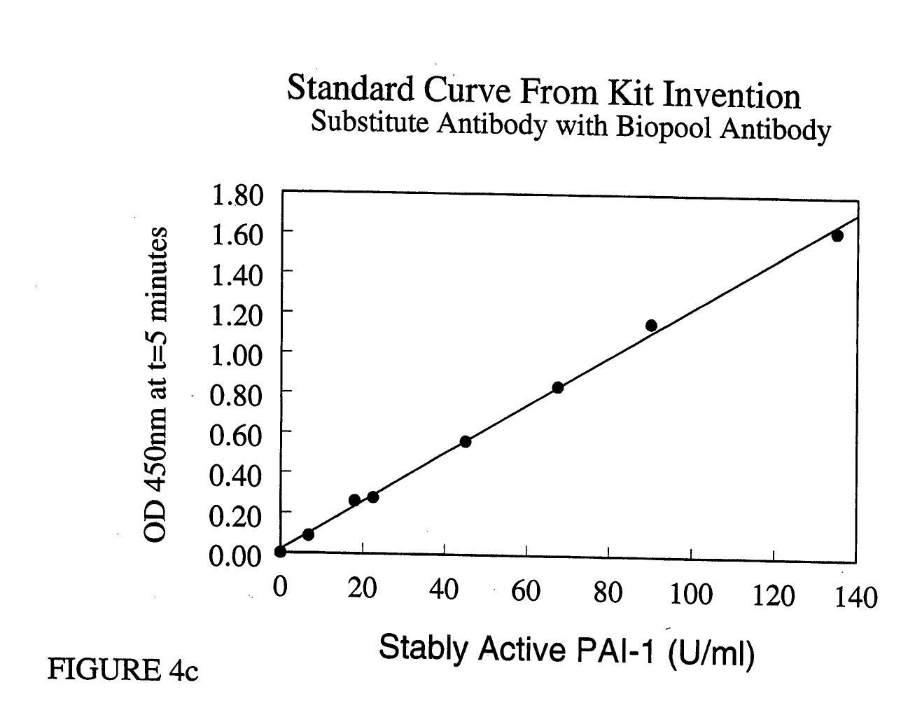

To determine the effect of using different primary antibodies in the assay, different monoclonal antibodies that recognize PAI-1 were used in the assay method described above. When two other monoclonal antibodies 671.3E5.29 and 671.2A7.8 (Molecular Innovations Inc.) were substituted for MA 33B8 (see Example 9), values of 26.5 U/ml and 27.6 U/ml resulted, respectively (FIG. 4a and FIG. 4b). Further, when Biopool's antibody was substituted in the present assay, a value of 28.9 U/ml was obtained (FIG. 4c). These results show a high degree of reproducibility for the calculated NIBSC standard using any of the above antibodies. The results further indicate that various different suitable primary antibodies can be used in the assay.

### Example 11

Comparative Data Set from the Biopool Chromolize® and STABLY ACTIVE PAI-1 Assays

Human blood was collected in sodium citrate (9:1 volume/volume), and plasma prepared by centrifugation in a refrigerated centrifuge at 4° C. The quantitative determination of active PAI-1 was made using the Biopool Chromolize® assay following the manufacturer's instructions. The STABLY ACTIVE PAI-1 assay was performed as described in Example 7. Eight different plasma samples were used for this comparison of the two assays. The data from these experiments clearly show a difference between the two systems in the detection of PAI-1 in human plasma (FIG. 5). The results demonstrate that 1) the stably active assay (i.e., assay using STABLY ACTIVE PAI-1) provides values that are comparable, yet distinctly different, from the Biopool assay, 2) the stably active assay consistently results in higher PAI-1 values than does the Biopool assay, 3) the stably active assay has the ability to determine higher values (concentrations) of PAI-1 in human plasma, as dictated by the larger range of the standard curve, and 4) the stably active assay is more sensitive than the Biopool assay as shown in Sample 6, where the Biopool assay detected a complete absence of PAI-1, yet the stably active assay detects approximately 2 U/ml of PAI-1 activity.

When ranges are used herein, such as molecular weight, or activity (e.g., as STABLY ACTIVE PAI-1 activity standard) all combinations and subcombinations of ranges specific embodiments therein are intended to be included.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method comprising
a) providing a STABLY ACTIVE PAI-1; and
b) establishing a standard curve using the STABLY ACTIVE PAI-1.

2. The method of claim 1, wherein the method further comprises comparing the amount of PAI-1 in a sample to the standard curve.

3. The method of claim 1, wherein the half-life of the STABLY ACTIVE PAI-1 is greater that the half-life of a wild type PAI-1.

4. The method of claim 1, wherein the half-life of the STABLY ACTIVE PAI-1 is at least two-fold greater than the half-life of a wild type PAI-1.

5. The method of claim 1, wherein the STABLY ACTIVE PAI-1 is derived from a wild type PAI-1 and comprises a mutation.

6. The method of claim 5, wherein the mutation is at least one of K154T, Q319L, M354I, N150H, and combinations thereof.

7. The method of claim 6, wherein the mutation is K154T, Q319L, M354I, and N150H.

8. The method of claim 1, wherein the sample is a biological sample.

9. The method of claim 1, wherein the sample is from a human.

10. The method of claim 8, wherein the biological sample is a body fluid.

11. The method of claim 10, wherein the body fluid is blood, plasma, or serum.

12. A method for determining the amount of active plasminogen activator inhibitor-1 (PAI-1) in a sample, the method comprising:
a) providing a sample;
b) contacting the sample with a PAI-1 binding molecule, thereby forming an active PAI-1 complex comprising a PAI-1 binding molecule and an active PAI-1;
c) separating the active PAI-1 complex from uncomplexed components; and
d) determining the amount of active PAI-1 in the sample by detecting the amount of the active PAI-1 complex and correlating the amount of the active PAI-1 complex to the amount of active PAI-1 using a standard curve.

13. A method for determining the amount of active PAI-1 in a sample, the method comprising
a) providing a sample;
b) contacting the sample with a PAI-1 binding molecule, and an anti-plasminogen activator-1 inhibitor antibody (anti-PAI-1) to form a PAI-1 complex comprising the antibody, active PAI-1 from the sample, and the PAI-1 binding molecule;
c) separating the PAI-1 complex from an uncomplexed components; and
d) determining the amount of active PAI-1 in the sample by detecting the amount of PAI-1 complex and correlating the amount of complex with the amount of active PAI-1 using a standard curve.

14. The method of claim 13, wherein the PAI-1 binding molecule is immobilized on an insoluble support.

15. The method of claim 14, wherein the PAI-1 binding molecule is immobilized directly onto an insoluble support.

16. The method of claim 14, wherein the PAI-1 binding molecule is chemically modified to be immobilized onto the insoluble support.

17. The method of claim 13, wherein PAI-1 complex is separated from unbound antibody prior to detecting the amount of PAI-1 complex.

18. The method of claim 13, wherein in step (b) a PAI-1 binding molecule/PAI-1 complex formed and is separated from unbound components prior to forming the PAI complex with the anti-PAI-1.

19. The method of claim 13, wherein the PAI-1 binding molecule is immobilized on an insoluble support via one or more linker molecules.

20. The method of claim 19, wherein the linker molecule comprises an antibody that can bind the PAI-1 binding molecule.

21. The method of claim 20, wherein the antibody is immobilized onto the insoluble support via a secondary linker molecule.

22. The method of claim 21, wherein the secondary linker molecule comprises a reporter group.

23. The method of claim 22, wherein the reporter group is selected from the group consisting of a radioisotope, a fluorescent group, a luminescent group, an enzyme, biotin, a dye particle, and combinations thereof.

24. The method of claim 19, wherein the secondary linker molecule is selected from the group consisting of avidin and biotin.

25. The method of claim 13, wherein detecting comprises using an enzyme-linked immunosorbent assay (ELISA), a Western blot, an immunohistochemical assay, an immunofluorescence assay, or an imaging assay.

26. The method of claim 12 or claim 13, wherein the PAI-1 binding molecule is a serine proteinase.

27. The method of claim 26, wherein the serine proteinase is a plasminogen activator.

28. The method of claim 27, wherein the plasminogen activator is a urokinase plasminogen activator.

29. The method of claim 27, wherein the plasminogen activator is a tissue plasminogen activator.

30. The method of claim 12 or claim 13, wherein the PAI-1 binding molecule is selected from the group consisting of a serine protease, tPA, uPA, vitronectin, glycosaminoglycan, fibronectin, cathepsin G, prostate specific antigen, and combinations thereof.

31. The method of claim 13, wherein the antibody is a monoclonal antibody.

32. The method of claim 13, wherein the antibody is a polyclonal antibody.

33. The method of claim 12 or claim 13, wherein the standard curve is established using a STABLY ACTIVE plasminogen activator inhibitor-1 (STABLY ACTIVE PAI-1).

34. The method of claim 33, wherein the half-life of the STABLY ACTIVE PAI-1 is greater that the half-life of a wild type PAI-1.

35. The method of claim 33, wherein the half-life of the STABLY ACTIVE PAI-1 is at least two-fold greater than the half-life of a wild type PAI-1.

36. The method of claim 33, wherein the STABLY ACTIVE PAI-1 is derived from a wild type PAI-1 and comprises a mutation.

37. The method of claim 33, wherein the STABLY ACTIVE PAI-1 is derived from a wild type PAI-1 and comprises at least one mutation that is K154T, Q319L, M354I, N150H, or a combination thereof.

38. The method of claim 33, wherein the STABLY ACTIVE PAI-1 mutation is K154T, Q319L, M354I, or N150H.

39. A method for determining the amount of active plasminogen activator inhibitor-1 (PAI-1) in a sample, the method comprising
a) providing a sample;
b) contacting the sample with a PAI-1 binding molecule, thereby forming an active PAI-1 complex comprising a PAI-1 binding molecule and an active PAI-1; and
c) determining the amount of active PAI-1 in the sample by detecting the amount of the active PAI-1 complex and correlating the amount of the active PAI-1 complex to the amount of active PAI-1 using a standard curve, and wherein the standard curve is established using a STABLY ACTIVE PAI-1.

40. A method for diagnosing a PAI-1 related disorder in a subject, the method comprising
a) obtaining at least one biological sample from a subject;
b) contacting the biological sample with a PAI-1 binding molecule to form an active PAI-1 complex comprising PAI-1 binding molecule and active PAI-1;
c) separating the active PAI-1 complex from a uncomplexed components; and
d) determining the amount of active plasminogen activator inhibitor-1 in the biological sample by detecting the amount of active PAI-1 complex in the sample and correlating the amount of complex to the amount of active PAI-1 using a standard curve.

41. The method of claim 40, wherein the subject is a human.

42. The method of claim 40, wherein the biological sample is a body fluid.

43. The method of claim 40, wherein the body fluid is blood.

44. The method of claim 40, wherein the body fluid is plasma.

45. The method of claim 40, wherein the PAI-1 binding molecule is a serine proteinase.

46. The method of claim 45, wherein serine proteinase is plasminogen activator.

47. The method of claim 46, wherein plasminogen activator is urokinase plasminogen activator.

48. The method of claim 46, wherein plasminogen activator is tissue plasminogen activator.

49. The method of claim 40, wherein the PAI-1 binding molecule is selected from the group consisting of serine proteases, tPA, uPA, vitronectin, glycosaminoglycan, fibronectin, cathepsin G, prostate specific antigen, and combinations thereof.

50. The method of claim 40, wherein the standard curve is established using a STABLY ACTIVE PAI-1.

51. A diagnostic kit comprising components for carrying out the method of claim 12.

52. A diagnostic kit comprising STABLY ACTIVE PAI-1.

53. The diagnostic kit of claim 52, further comprising instructions for preparing a standard curve.

54. A diagnostic kit comprising
a) PAI-1 binding molecule;
b) at least one anti PAI-1 binding molecule antibody;
c) at least one detection reagent; and
e) STABLY ACTIVE plasminogen activator-1 inhibitor.

55. The kit of claim 54, wherein the kit further comprises at least one buffer and instructions for use of the kit.

56. The kit of claim 54, wherein the PAI-1 binding molecule is a serine protease.

57. The kit of claim 54, wherein the kit further comprises an insoluble support.

58. The kit of claim 54, wherein the PAI-1 binding molecule in supplied on an insoluble support.

59. A method of identifying an agent that can modulate active PAI-1, the method comprising,
a) providing a test agent;
b) determining whether the test agent can modulate the activity of PAI-1 using the method of claim 12; and
c) selecting a test agent that modulates the activity of PAI-1.

60. The method of claim 60, wherein a STABLY ACTIVE PAI-1 is used to establish the standard curve.
